(19) United States
(12) Patent Application Publication
(10) Pub. No.: US 2025/0257371 A1
(43) Pub. Date: Aug. 14, 2025

(54) Method of Reducing Water Consumption in Bioethanol Production Process

(71) Applicant: G2B BioSolutions ApS, Hellerup (DK)

(72) Inventors: Dayanand Jadhav, Hellerup (DK); Prashant Madhusudan Bapat, Hellerup (DK)

(21) Appl. No.: 18/859,243

(22) PCT Filed: May 4, 2023
(86) PCT No.: PCT/EP2023/061838

§ 371 Date: Oct. 23, 2024

(30) Foreign Application Priority Data
May 5, 2022 (DK) PA202270237

(51) Int. Cl. C12P 7/14; C12F 3/02; C12M 1/00; C12R 1/07; C12R 1/25

(52) U.S. Cl. CPC C12P 7/14; C12F 3/02; C12M 29/00; C12R 2001/07; C12R 2001/25

(57) ABSTRACT

The present invention provides a method for making ethanol and a protein feed or food product from a feedstock including starch, such as grain and grain-derived products, preferably combined with CO₂ capture to increase yields further. The method facilitates a reduction in water usage compared to traditional ethanol plants, without affecting quality and quantity of the end products.

### CROSS REFERENCE TO RELATED APPLICATIONS

The present application is a § 371 national stage of international application no. PCT/EP2023/061838, filed on May 4, 2023, which claims priority to DK application no. PA202270237, filed May 5, 2022, the contents of both of which are incorporated by reference herein.

### FIELD OF THE DISCLOSURE

The present invention provides a method for making ethanol and a protein feed or food product from grain and grain-derived products, preferably combined with CO2 capture to increase yields further. The method facilitates a reduction in water usage compared to traditional ethanol plants, without affecting quality and quantity of the end products.

The present invention relates to a method for producing ethanol and a high protein feed or food product from a feedstock comprising starch, where key steps of the method comprise bio-preservation of feedstock using probiotic treatment, raw starch hydrolysis, use of thin stillage for steam production, reuse of process water, CO2 capture using carbonic anhydrase.

By combining several process steps in a novel and innovative way, the method of the present invention facilitates conservation of the moisture from the feedstock material and related water savings, reduction in overall water consumption in the ethanol production plant, reduced ethanol loss as a result of less evaporation, overall improved ethanol yields, removal of volatile acids in backset water, improved conditions for yeast fermentation.

### BACKGROUND

International organizations agree that achieving carbon neutrality by 2050 will require an increased uptake of sustainable biofuels like bioethanol, an ethanol is produced from biological sources by fermentation using microorganisms such as yeasts. During the last few years, bioethanol has been responsible for avoiding the release of more than 500 million tons of CO₂ in the atmosphere. The production of bioethanol is therefore encouraged and its market as a biofuel is likely to grow even more in future. However, by 2030, the world is going to face almost 40% gap in supplying fresh water. This might become a potential bottleneck towards sustainable bioethanol production bringing a new urgency to addressing water scarcity.

As per DOE report published in 2019 (Wu M. & Xu. H, 2018), atypical well optimized dry milled first generation ethanol plant (i.e. starch based ethanol plants) consumes 2.8-l. water to produce one-l. ethanol (3.15 l water is evaporated in cooling tower whereas corn moisture brings ˜0.3-liter water, resulting in 2.8-l. water per l. ethanol). Most of this water is required primarily for heating, scrubbing, cooling, and drying. It is important to note that most of these ethanol plants have little or no wastewater discharge. They recycle a significant portion of their process water through a backset. Therefore, water demand primarily is related to energy production, specifically the cooling tower and boiler systems. As per analysis conducted by Pfromm. P, 2008, a conventional dry milled ethanol plant consumes around 9194 BTU (British Thermal Units) energy to produce one-liter ethanol. The main reason behind such a high energy consumption is hot liquefaction process where starch from the grain is hydrolyzed to sugars at 85° C. The thermal energy intake poses a challenge towards reducing water consumption further as the fundamental fact is, that thermal energy intake of the plant must be balanced by an appropriate heat sink, which is evaporation of water in a cooling tower.

Another way of hydrolyzing starch is by raw starch hydrolysis or cold mashing, where the liquefaction temperature is below the starch gelatinization temperature ˜45-50° C. The cold mashing process (as described in Lewis et. al., 2011; U.S. Pat. No. 874,814B2) can potentially reduce thermal energy intake. In the cold mashing process, starch is enzyme hydrolyzed at <50° C. resulting in significant reduction in energy intake (˜4732 BTU/ethanol, based on mass balance). As per thermodynamic principle, this should reduce water consumption significantly to 1.6-l. water/l. ethanol (based on energy balance). However, the realized water consumption is at ˜2.2 l. water/l. ethanol (1.6 l water is evaporated in cooling tower +1.5-liter fresh water required in process −0.3 l water from corn moisture −0.6 l water saved on boiler). The main limitation of the cold mashing process is its inability to recirculate backset due to high concentration of volatile acids (VAs: lactic acid and acetic acid)._The backset containing >5 g/l VAs can inhibit yeast and result in sluggish/stuck fermentation. Further, as cold mashing avoids jet cooking (105-121° C., for 5-10 min) and hot liquefaction (85° C. for 2 h), it is prone for contamination from lactic acid bacteria (LAB), acetic acid bacteria (AAB) and molds. The possible presence of such potentially harmful contaminants is another reason why very few ethanol plants use cold mashing technology. Plants using cold mashing are aware of this and the way they circumvent this challenge is by:
a) adjusting pH in the range of 3.0-4.5 using inorganic acids (for example: sulfuric or phosphoric acid)
b) adding antibiotics or hops acids in the saccharification/fermentation stage
c) sending thin/whole stillage to an anaerobic digester (AD) facility.

Both, conventional and cold mashing technologies need to use dried feedstock (˜16% moisture w/w). Typical moisture content of fresh feedstock is ˜30%, which can potentially support microbial contamination, for example mold as well as LAB and AAB. Mold infection may give rise to mycotoxins resulting in bad quality feed product (Distiller's dried grains with solubles, DDGS) unsuitable for consumption by livestock such as ruminating animals and pig, thus a big risk of revenue loss for bioethanol producers. LAB and AAB infection can also result in sluggish or stuck ethanol fermentation. Most of the ethanol producers avoid this by feedstock drying (removing moisture from the grain using hot air). The dried feedstock has moisture content ˜16%, meaning that almost 14% water or ˜0.3 l. water/l. ethanol is lost during drying process (Pfromm. P, 2008). Apart from loss in water, many times, harsh drying is also responsible for reduced grain quality in terms of starch retro gradation, lower oil and protein yield. Moreover, drying process also results in increased carbon footprint on incoming feedstock.

To prevent LAB and AAB buildup, plants operating in batch mode (using both conventional and cold mashing technologies) need to clean heat exchangers with hot (˜90° C.) sodium hydroxide (5% NaOH) every day. However hot caustic treatment is not full proof, many times these contaminants form biofilms (Rich et al., 2015) rendering clean in place (CIP) treatment ineffective and resulting in sluggish fermentation. Ethanol manufacturers overcome this by increasing CIP treatment duration and/or using harsh chemicals such as Nitric acid. To prevent air pollution due to volatiles (VOCs) such as acetaldehyde, ethyl acetate, acrolein and acetone, most of the ethanol plants install scrubbers in the vent on the fermenters. Fresh water is circulated in scrubbers and mixed with a variety of additives to wash CO₂ and to trap VOCs adding its share to water footprint.

### SUMMARY

The present invention provides a method for making ethanol and a protein feed or food product, from feedstock comprising starch and protein, said method comprising the following steps of:
providing a feedstock comprising starch,
pretreating the feedstock by applying a probiotic microorganism,
adding an aqueous liquid to the pretreated feedstock, followed by mixing to obtain a slurry,
optionally adjusting pH of the slurry to 5-7, such as by addition of ammonia water,
enzymatically treating the slurry by addition of an amylase enzyme, to obtain a hydrolysate,
fermenting the hydrolysate by addition of yeast, and
separately recovering
ethanol
CO₂, and
protein.

In one embodiment, the present invention provides a method for making ethanol and a protein feed or food product, from feedstock comprising starch and protein, said method comprising the steps of:
providing the feedstock comprising starch and protein,
pretreating the feedstock by applying a culture of one or more probiotic species of Lactobacillus and/or spore forming Bacillus to the feedstock,
adding an aqueous liquid to the pretreated feedstock, followed by mixing to obtain a feedstock slurry,
optionally adjusting pH of the feedstock slurry to pH 5-7,
adding an amylase enzyme to the feedstock slurry and incubating the slurry to obtain a feedstock hydrolysate,
adding yeast to the feedstock hydrolysate to obtain a fermentation broth and fermenting the fermentation broth by continuous fermentation comprising two stages, wherein stage 1 has a dilution rate between 0.10-0.55 h⁻¹ and stage 2 has a dilution rate between 0.04-0.10 h⁻¹, resulting in a steady state ethanol concentration between 5-11% (w/w) in stage 1 and between 10-13% (w/w) in stage 2, and
separately recovering
ethanol,
CO₂, and
protein,
wherein CO₂ is captured in the fermentation broth during the continuous fermentation by addition of carbonic anhydrase and Zn⁺ to the fermentation broth; and wherein ethanol and CO₂ are separated and recovered in a distillation and condensation step.

In one embodiment, the feedstock is grain and/or one or more grain derived products, such as grain selected from wheat, rice, oats, barley, rye, barley, millet, corn, triticale, and sorghum grain.

In one embodiment, the water content of the feedstock is at least 10% w/w, at least 15% w/w, or at least 20% w/w, or at least 30% w/w.

In one embodiment, the probiotic culture in step (b) is applied onto the surface of the feedstock by spraying the probiotic culture on the surface, and optionally mixing.

In one embodiment, the probiotic culture in step (b) comprises a species of Lactobacillus and a species of spore forming Bacillus; such as a species of Lactobacillus selected from Lactobacillus reuteri, Lactobacillus brevis, Lactobacillus plantarum, Lactobacillus amylovorus, and Lactobacillus hammesii; such as a species of spore forming Bacillus is selected from Bacillus licheniformis, Bacillus clausii, Bacillus subtilis, Bacillus amyloliquefaciens, Bacillus fusiformis and Bacillus megaterium.

In one embodiment, the continuous fermentation in step (f) comprises an additional third stage having a dilution rate between 0.04-0.10 h⁻¹, resulting in an ethanol concentration in stage 3 between 12-15% (w/w).

In one embodiment, step (f) of the method comprises maintaining a pH of between 5-6 in fermentation stage 1 and stage 2.

In one embodiment, step (g) of the method comprises addition of 0.3×10⁻¹² to 3.0×10⁻¹² mol Zn⁺ per unit of carbonic anhydrase.

In one embodiment, Zn⁺ is added in the form of ZnSO₄.

In one embodiment, the recovery and separation of the ethanol and the CO₂ comprises vaporizing the ethanol and CO₂, separating the vapors of the ethanol and the CO₂ using a condenser, where the ethanol is condensed in liquid form and the CO₂ is liberated in gaseous form; such as vaporizing the ethanol and CO₂ is at a temperature of 70-90° C., and condensing the ethanol at a temperature of 5-30° C.

In one embodiment, step (g) of the method further comprises recovering an aqueous liquid resulting from one or more preceding steps of the method, such as from the distillation step; and wherein the aqueous liquid added to the feedstock in step (c) comprises the comprises the aqueous liquid recovered in step (g), such as more than 50% of the aqueous liquid added to the feedstock in step (c) being the aqueous liquid recovered in step (g).

### BRIEF DESCRIPTION OF THE FIGURES

FIG. 1A is an illustration of a typical dry milled bioethanol production plant, comprising the features and process steps (a) slurry mixing tank, (b) jet cooker, (c) liquefaction tank, (d) yeast propagation tank, (e)-(h) battery of batch fermenters, (i) beer well tank, (j) distillation column, (k) molecular sieves, (l) Ethanol storage tank, (m) CO2 purification system, battery of three compressors, (n) CO₂ storage tank, (o) decanter/centrifuge, (p) thin stillage storage tank, (q) evaporator effects, (r) syrup tank, and (s) DDGS dryer.

FIG. 1B shows G2B's Sustain^{Max} process, where innovation steps 1-7 (marked with stars) result in significant reduction in energy and water consumption. Innovation step 1: Pretreatment of grain material with probiotic culture; Innovation step 2: Adjustment of slurry pH with ammonia water and cold liquefaction (raw starch hydrolysis) is performed; Innovation step 3: Continuous fermentation process, dosing Carbonic anhydrase and addition of Ammonium nitrate; Innovation step 4: Releasing CO2 at distillation stage; Innovation step 5: Boiling stillage to make steam vapors for distilling ethanol; Innovation step 6: Separation of fibers from whole stillage; Innovation step 7: Hydrocyclone-based separation of insoluble proteins from filtrate. The process comprises the features and steps (A) slurry mixing & cold mashing tank, (B) yeast propagation tank, (C)-(E) continuous reactors stage 1, 2, and 3, respectively, (F) stillage boiling tank, (G) Distillation column, (H) Condenser, (I) CO₂ purification system compressor, (J) CO₂ storage tank, K) molecular sieves, (L) ethanol storage tank, (M) separation, (N) filtrate, (O) Hydro cyclone, and (P) drying.

FIG. 2 is an illustration of three stage continuous fermentation system. Liquefied slurry from liquefaction (hydrolysis) tank is continuously fed to stage 1 fermenter using pump P1. The flow rate is adjusted to maintain dilution rate in the range of 0.1-0.55 h⁻¹. pH in stage 1 fermenter is continuously measured using pH probe. The pH is maintained between 5.5-7.0 using base. Pump P2 continuously pumps out fermented mash from stage 1 fermenter and doses into stage 2 fermenter. The flow rate is adjusted to maintain dilution rate in the range of 0.04-0.1.

FIG. 3 is a plot of ethanol percentage at different stages of a process.

### DETAILED DESCRIPTION

Abbreviations, terms, and definitions:

As used herein, the phrase “cold mashing” refers to a process for converting starch to ethanol using alpha-amylase for saccharification of the starch and without heat treatment for gelatinization. Generally, for the process of the present invention, “cold mashing” refers to maintaining a temperature below starch gelatinization temperatures, so that the enzymatic saccharification occurs directly from the raw native insoluble starch to soluble glucose while bypassing conventional starch gelatinization conditions. Starch gelatinization temperatures are typically in a range of 50° C. to 93° C. depending on the starch source and polymer type. In the method of the present invention, dextrinization of starch using conventional liquefaction techniques is not necessary for efficient fermentation of the carbohydrate in the grain.

As used herein, the phrase “feedstock” refers to all feedstock material comprising starch (e.g., cereal grain, bread, bakery products). The feedstock further comprises protein. Preferably the feedstock is a grain and/or grain-derived product. Suitable feedstocks include bakery products like bread, croissant, dough, biscuits, cake, and grains such as maize (corn, e.g., whole ground corn), Sorghum (milo), barley, wheat, rye, rice, and millet; and Starchy root crops, tubers, or roots such as Sweet potato, cassava. The feedstock can be a mixture of such materials.

As used herein, the phrase “probiotics” refers to bacteria and yeast, which are considered generally safe to consume. Suitable probiotics includes lactic acid bacteria, such as Lactobacillus plantarum JA7, W. anomalus LCF1695, Lactobacillus amylovorus, and Lactobacillus hammesii. Other suitable probiotics include Bacillus species such as Bacillus licheniformis, Bacillus clausii, Bacillus subtilis, Bacillus amyloliquefaciens, Bacillus fusiformis, and Bacillus megaterium (also called as Priestia megaterium). The probiotics can be mixture of bacteria and yeast.

As used herein, the phrase “baseline” refers to existing state of the art bio-ethanol production process.

As used herein, the phrase “High Protein” refers to the protein co-product obtained from an ethanol fermentation process, wherein said “high protein” product comprises at least 50% protein (at ˜10% moisture).

The term “disposal” as used herein refers to grain and/or grain derived products declared as not suitable for human consumption.

The term “liquefact” as used herein refers to the grain hydrolysate obtained from the hydrolysis step. The term “CIP” as used herein refers to Clean In Place.

The term “whole stillage” as used herein refers to the aqueous fraction left after removal of ethanol during the distillation stage.

The term “wet cake” as used herein refers to insoluble portion obtained after centrifugation of the whole stillage.

The term “thin stillage” as used herein refers to the aqueous fraction left after removing the wet cake after centrifugation of the whole stillage.

The term “DDGS” as used herein refers to Distiller's dried grains with solubles.

The terms “backset” or “backset water” or “backset liquid” as used herein refers to a mixture of thin stillage and water produced during DDGS drying, molecular sieves and evaporator operations.

The term dilution rate (D) as used herein, usually expressed in units per hour (h⁻¹), refers to the relationship between the flow of medium into a fermenter (liters h⁻¹), and the fermentation volume within the fermenter (liters). The term “ammonia water” as used herein is a solution of ammonia in water, commonly denoted by the symbols NH₃(aq)

The present invention facilitates a reduction in water usage and energy consumption compared to traditional ethanol plants, without affecting quality and quantity of the end products. It provides a novel method for making ethanol and a protein feed or food product from grain and grain-derived products, preferably combined with CO₂ capture to increase yields further. The overall process is highly sustainable with ultra-low GHG (green-house-gas) emissions and robust against large fluctuations in moisture content of the feedstock.

The present invention relates to a method for producing ethanol and a high protein feed or food product, where key steps of the method comprise bio-preservation of feedstock using probiotic treatment, raw starch hydrolysis, continuous fermentation process, use of thin stillage for steam production, reuse of process water and reclaim and recycle of backset and process water close to 100%.

By combining several process steps in a novel and innovative way, the method of the present invention facilitates conservation of the moisture from the feedstock material and related water savings by avoiding feedstock drying, reducing water consumption in cooling tower, significant inclusion of backset such as up to 100%, significant reduction in CIP (clean in place) cycles, avoiding mold contamination in feedstock, avoiding antibiotic addition in saccharification and/or fermentation stage, and improves conditions for yeast fermentation.

### I. Method for Making Ethanol and a Protein Feed or Food Product

The present invention provides a method for making ethanol and a protein feed or food product, which may additionally be combined with CO₂ capture to increase yields further.

In one embodiment, the method comprises the following steps of:
providing grain and/or grain-derived products,
pretreating the grain and/or grain-derived products by applying a probiotic microorganism to the grain and/or grain-derived products,
adding an aqueous liquid to the pretreated grain and/or grain-derived products, followed by mixing to obtain a grain slurry,
optionally adjusting pH of the grain slurry to ≥5.5 by addition of ammonia water to the grain slurry,
enzymatically treating the grain slurry by addition of an amylase enzyme to the grain slurry to obtain a grain hydrolysate,
fermenting the grain hydrolysate by addition of yeast to the grain hydrolysate, and
separately recovering
ethanol
protein, and
optionally CO₂

In one embodiment, the method comprises the following steps of:
providing a feedstock comprising starch,
pretreating the feedstock by applying a probiotic microorganism,
adding an aqueous liquid to the pretreated feedstock, followed by mixing to obtain a slurry,
optionally adjusting pH of the slurry to 5-7, such as by addition of ammonia water,
enzymatically treating the slurry by addition of an amylase enzyme, to obtain a hydrolysate,
fermenting the hydrolysate by addition of yeast, and
separately recovering
ethanol
CO₂, and
protein.

The present invention improves water reclamation in an ethanol production plant, especially in a dry milled ethanol production plant. The invention provides a novel process for producing bioethanol, based on the traditional cold mashing process, but with the biomass feedstock initially being treated with probiotic microorganisms to ensure no unwanted bacterial or fungal growth, and then thereafter hydrolyzed preferably using cold mashing process, and finally used to ferment ethanol preferably in continuous mode with specific stage related dilution rates. In this way, total water consumption is significantly decreased, which can be achieved without requiring any advanced chemical separation technology or ion exchange resin-based process. The applicability of said probiotic treated feedstock is further improved by adding digestate (ammonia water) from an anaerobic digester (biogas) plant before the cold mashing process. Running ethanol fermentation process in continuous mode while maintaining stages at specific dilution rates resulting in contamination prevention and ethanol yield similar to baseline. Finally, a CO₂ capturing enzyme is preferably added in the fermentation step resulting in reducing water loss further. In addition to recovering ethanol from the process, a protein product is also recovered, which is essentially ‘pure’ and can therefore be used as feed or in food applications.

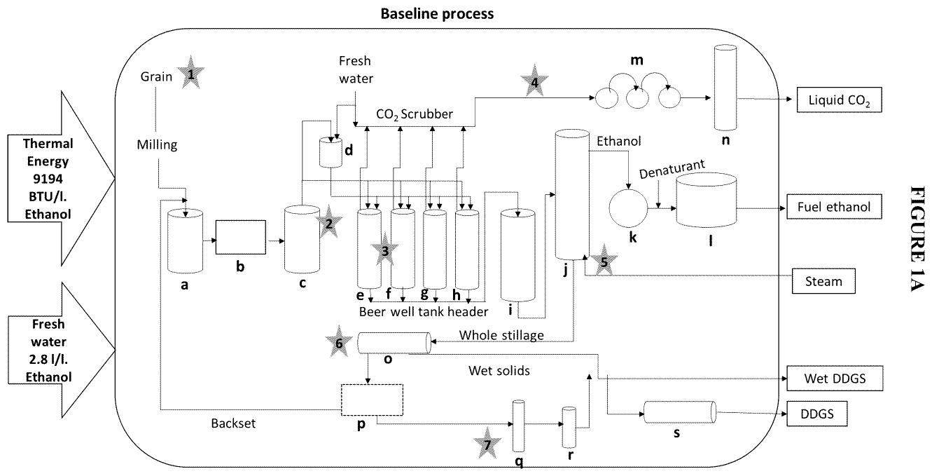

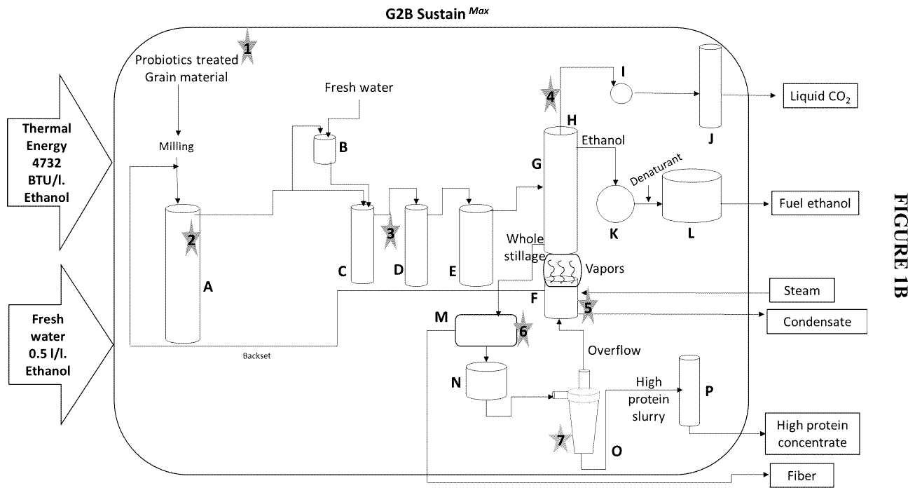

The different process steps are described in greater detail below. The present invention is graphically illustrated in FIG. 1B. The illustration should not be seen as limiting for the invention but merely provides an illustration of one means of performing the invention to produce ethanol a protein feed/food product, and CO₂, while reducing energy and water consumption. For comparison, FIG. 1A illustrates the ‘baseline’ typical dry milling bioethanol production plant.

### I.i Feedstock—Such as Grain and Grain-Derived Products

The present invention provides a method for making ethanol and a protein feed or food product from feedstocks comprising starch. In one preferred embodiment, the feedstock is grain and/or grain-derived products.

“Grains” are the edible seeds of plants. There are two main types of grain crops as cereals and legumes. Cereal grains are members of the Poaceae family and contain a high carbohydrate content. Wheat, rice, oats, barley, rye, barley, millet, corn, triticale, and sorghum are some examples of cereal grains. Grains also include pseudo-cereals like chia, quinoa, and buckwheat gains. Legumes or pulses are members of the pea family and they have a higher protein content than cereal grains. Chickpeas, mung beans, soybeans, common beans, lentils, and lima beans are some further examples of grains.

“Grain-derived products” refers to products made using above mentioned grains and may comprise of high starch content. Flours made from above mentioned grains are examples of grain derived products. Bread, cakes, dough, chocolates, biscuits, breakfast cereals are further some examples of grain derived products.

“Grain material” is used herein as a common term for grain and grain-derived products.

In one embodiment, the grain material has a starch content of at least 40, 45, 50, 55, 60, 65, 70, 75, 80, 85, 90% or even at least 95%. In one embodiment, the grain material has a starch content between 5-95%, 10-95%, 15-95%, 20-95%, 25-95%, 30-95%, 35-95%, 40-95%, 45-95%, 50-95%, 55-95%, 60-95%, 65-95%, 70-95%, 75-95%, 80-95% or 90-50%.

In one embodiment, the grain material is selected from wheat, rice, oats, barley, rye, barley, millet, corn, triticale, and sorghum grain, and any product derived from these grains. In a preferred embodiment, the grain material is bread derived from wheat.

I.ii Pretreatment Using Probiotic Bacteria and/or Yeast

In one aspect of the invention, a probiotic microbe is applied to the feedstock material (such as grain material) in order to enhance microbial stability and reduce unwanted mold contamination in the feedstock material. The term pretreatment implies that the step takes place before the hydrolysis and fermentation steps. In FIG. 1, this is illustrated as “Innovation step 1” (star 1). The positive effect of probiotic microbial pretreatment is further demonstrated in Example 1.

Traditionally, the feedstock would be dried or have its water content reduced by other means to ensure a less favorable environment for unwanted contaminants. Meanwhile, for the present invention, such drying step may be omitted or at least significantly reduced due to the probiotic microbes preventing the growth of other unwanted microbial contaminants. The potential problem of contamination mostly arises when the provision of the feedstock material includes a ‘storage step’, such as the time required to transport the feedstock material to the ethanol plant from its origin, or during other general ‘storage’ conditions prior to its use in the present process.

In one embodiment, probiotic microbes are added to feedstock material having a moisture content (i.e. water content) greater than 5, 10, 15, 20, 25, 30, 35, 40 or even greater than 45%. In one embodiment, probiotic microbes are added to feedstock material having a water content between 10-50%, 15-50%, 20-50%, 25-50%, 30-50%, 40-50%, or 45-50% (w/w). In one embodiment, probiotic microbes are added to feedstock material having a water content between 10-15%, 10-20%, 10-25%, 10-30%, 10-35%, 10-40%, or 10-45% (w/w). In one embodiment, probiotic microbes are added to feedstock material having a water content between 10-40%, preferably between 15-40%, more preferably between 20-30% (w/w).

An advantage of the present invention is that it provides a method, wherein the grain material can be used ‘as is’, such as without decreasing the water content of the feedstock material. The microbial pretreatment is a solution towards enhancing shelf life of the feedstock, without decreasing its water activity as well as without decreasing nutritional properties.

As illustrated in Example 8, the water contribution within the ethanol process from the feedstock material may be increased by as much as 50% as a result of not needing to dry the grain material in a drying process (see further calculation details in Example 8).

In one embodiment, the water contribution from the feedstock is increased by 5, 10, 15, 20, 25, 30, 35, 40, 45, or even by 50% within the ethanol process, compared to the traditional process, where drying of the feedstock is required. Probiotic bacteria will not only reduce and/or prevent bacterial and mold contamination in the initial feedstock material, but will also result in reducing contamination in the later ethanol fermentation. In one embodiment, the probiotic microbe is a microbial cell or cell culture selected from a bacteria or a yeast, or is a combination of a bacteria and a yeast.

In one embodiment, the probiotic microbe to be applied to the feedstock comprises one or more probiotic bacteria—i.e. one or more probiotic bacterial cells or probiotic bacterial cell cultures. In a preferred embodiment, the probiotic bacteria comprise one or more lactic acid bacteria. In one embodiment, the probiotic bacteria comprise one or more homofermentative lactic acid bacteria (HoLAB). In one embodiment, the lactic acid bacteria is one or more strain of the genus Lactobacillus. In one embodiment, the lactic acid bacteria are selected from Lactobacillus reuteri, Lactobacillus brevis, Lactobacillus plantarum (for example strain JA7), Lactobacillus amylovorus, and Lactobacillus hammesii. In one embodiment, the probiotic bacteria is one or more strain of the genus Pediococcus. In one embodiment, the probiotic bacteria is selected from Pediococcus acidilactici (for example strain KTU05-7), Pediococcus pentosaceus (for example strain KTU05-8) and Pediococcus pentosaceus (for example strain KTU05-10).

In one embodiment, the probiotic microbe to be applied to the feedstock comprises one or more yeast. In one embodiment, Pichia anomala (for example strain SKM-T) is used in the pretreatment of the feedstock material.

In one specific embodiment, the combination of probiotic microbes Lactobacillus plantarum and Wickerhamomyces anomalus are used in the pretreatment of the feedstock material. Specifically strains Lactobacillus plantarum JA7 and Wickerhamomyces anomalus LCF1695 may be used in the pretreatment of the feedstock material.

In one specific embodiment, a combination of spore forming probiotic microbes Bacillus licheniformis, Bacillus clausii, Bacillus subtilis, Bacillus amyloliquefaciens, Bacillus fusiformis and Bacillus megaterium (also called Priestia megaterium) may be used in the pretreatment of feedstock material.

In one preferred embodiment, the probiotic microbes applied in the method of the present invention comprise a combination of bacillus and lactobacillus species.

In one preferred embodiment, a culture/suspension of one or more probiotic species of Lactobacillus and/or spore forming Bacillus is applied to the feedstock.

In one such embodiment, the probiotic bacteria comprise of combination of (i) one or more strains of lactic acid bacteria disclosed herein, such as selected from Lactobacillus reuteri, Lactobacillus brevis, Lactobacillus plantarum (for example strain JA 7), Lactobacillus amylovorus, Lactobacillus hammesii, Pediococcus acidilactici (for example strain KTU05-7), Pediococcus pentosaceus (for example strain KTU05-8) and Pediococcus pentosaceus (for example strain KTU05-10), and (ii) one or more strains of Bacillus species as disclose herein, such as selected from Bacillus licheniformis, Bacillus clausii, Bacillus subtilis, Bacillus amyloliquefaciens, Bacillus fusiformis and Bacillus megaterium spore forming bacillus.

In one important embodiment, the microbial pretreatment of the feedstock material is performed by applying the probiotic microbes onto the surface of the feedstock material, such as on the surface of a grain material to avoid contamination by other unwanted microbial contaminants.

In one embodiment, the probiotic microbes are applied by spraying them on the surface of the feedstock material. As an example, the probiotic microbes may be sprayed onto bread to avoid contamination prior to the bread material undergoing the further process steps in the ethanol production facility.

In one embodiment, the probiotic microorganisms are applied onto the surface of the grain immediately after harvesting and/or the grain-derived product immediately after disposal, such as by spraying the microorganism on the surface.

In one embodiment, the probiotic bacteria and/or yeast are applied, and the grain and grain material is then milled to flour in a milling process step prior to the cold mashing step.

### I.iii Hydrolysis

The grain material is hydrolyzed in an aqueous solution. In FIG. 1, this is illustrated as “Innovation step 2” (star 2). Specifically, it is the pretreated feedstock (probiotic pretreatment as discussed in section Iii), preferably pretreated grain material, which is enzymatically hydrolyzed to obtain monomeric sugars for ethanol fermentation.

The feedstock may be chopped, milled or broken down/reduced in size by such known processes, before addition of the aqueous solution. In one preferred embodiment, the feed stock is milled to flour prior to the hydrolysis step.

Depending on the initial water content of the grain material, an aqueous solution is added to the grain material to obtain a grain slurry which has a desired dry matter percentage.

In one embodiment, hydrolysis is performed at a dry matter content between 20-40%, preferably between 25-35%, more preferably between, 28-33%.

In one embodiment, the method comprises recovering process water, where the aqueous liquid added to the feedstock to obtain the feedstock slurry, comprises the recovered process water. In one embodiment, the aqueous liquid added to the grain material comprises backset liquids from within the ethanol processing plant. This recycled water may come from the ethanol distillation process, illustrated as “Innovation step 5” (star 5) in FIG. 1B. In one embodiment, the aqueous liquid comprises a mixture of backset and fresh water.

The pH of the feedstock slurry is optionally adjusted to pH ≥5.5 before hydrolysis. In one embodiment, the feedstock is grain or a grain derived product, and the pH of the feedstock is adjusted to pH ≥5.5 before hydrolysis. In one embodiment, the pH of the feedstock is adjusted to pH 5-7, such as pH 5.5-7 before hydrolysis. In one such embodiment, the pH of the hydrolysis solution is adjusted by use of any alkali, such as an alkaline solution, e.g. potassium hydroxide, sodium hydroxide, or ammonia water. In a preferred embodiment, the pH is adjusted using ammonia water, such as disclosed in section Liv.

In the present method, the grain material is hydrolyzed at a temperature between 30-65 degree C., preferably a temperature between 35-65 degree C., more preferably between 40-55 degree C., most preferably between 40-45 degree C., even more preferably between 50-55 degree C. The temperature of the grain material may be controlled by adding process water as described above.

An advantage of the present invention is that it provides a method, wherein the conventional liquefaction for conversion of starch to sugars is instead performed by a cold mashing process, wherein the temperature is maintained below starch gelatinization temperatures and the enzymatic hydrolysis happens directly on raw (non-gelatinized) starch material. An example of such cold mashing process is provided in Example 3.

The grain material is hydrolyzed at a pH between 4-7, preferably a pH between 5-6.50, more preferably between 5.5-6.0. In some embodiments, pH adjustment is required at one or more time points during the hydrolysis to maintain the pH within these pH ranges.

Mixing is preferably performed during the hydrolysis process.

Starch hydrolyzing enzymes are used for hydrolysis of the starch in the grains. One or more amylase enzymes are used for hydrolysis of the starch in the grains. In one embodiment, the amylase enzymes comprise alpha- and/or beta-amylases belonging to the E.C. 3.2.1 enzyme classification.

In a further embodiment, a mixture of one or more of glucoamylase, acid amylase and cellulase are used in the hydrolysis step; these may also be used in combination with the alpha-amylase.

In one specific embodiment, the commercially available enzymes Liquoflow® GO 2× and Saczyme® (from Novozymes A/S) are used either alone or in combination.

Based on the feedstock for hydrolysis, dosing of the enzymes may be optimized as recognized by a person skilled in the art.

In one embodiment, an amylase enzyme is added to the feedstock slurry and the slurry is incubated to obtain a feedstock hydrolysate.

### I.iv Use of Ammonia Water for pH Adjustment

As recognized by a person skilled in the art, different process steps may require pH adjustment for optimal performance. In one embodiment, ammonia water is used for pH adjustment in the process of the present invention. The term “ammonia water” refers to an aqueous solution comprising ammonia. An example of pH adjustment using ammonia water is provided in Example 2.

One of the by-products of biogas plants is ammonia containing water. Disposing this water poses a big challenge to biogas plants; the main reason being the high ammonia emissions. Currently available ammonia extraction technologies are not only expensive but also comes with high carbon foot print. Therefore, in a preferred embodiment, the ammonia water originates from a biogas plant, such as derived from a biogas waste stream.

To this end, the present invention uses this ammonia water in the slurry mixing step in the ethanol process. This is illustrated in FIG. 1, as part of “Innovation step 2” (star 2).

Ammonia water adds more value than being a simple means of pH adjustment—it further also serves as a relevant source or nitrogen for the yeast in the fermentation step, compared to e.g. using simply hydroxide compounds for the pH adjustment.

### I.v Fermentation

The hydrolyzed feedstock material is fermented by yeast, such as Baker's yeast Saccharomyces cerevisiae. In FIG. 1B, this is illustrated as “Innovation step 3” (star 3).

In one embodiment, fermentation is performed at a dry matter content between 20-60%, preferably between 20-50%, more preferably 25-40%, most preferably between 28-32%.

Mixing is preferably performed during the fermentation process.

In one embodiment, the hydrolyzed grain material is fermented at a temperature between 20-40 degree C., preferably a temperature between 28-36 degree C., more preferably between 30-32-degree C.

In one embodiment, the fermentation is performed at a pH between 4-7, preferably a pH between 4.5-6.5, more preferably between 5.0-6.0, most preferably between 5.0-5.5; such as at a pH of 5.0, 5.1, 5.2, 5.3, 5.4 or 5.5. Keeping the pH above 5.0 will ensure that the lactic acid and acetic acid present in the solution will not inhibit yeast.

pKa (the acid dissociation constant) plays important role in yeast growth. Among all contamination reported in bio-ethanol process, acetic and lactic acid contamination is the most detrimental to yeast health. The volatile acids (VAs) are in protonated form below their pKa (pKa of acetic acid is 4.76; pKa of lactic acid is 3.86). At protonated state these VAs can pass through yeast cell membrane resulting in lowering cytoplasm pH. Yeast expels these VAs at the expense of ATP. However due to protonated form, these VAs keep on passing through yeast cell membrane barrier. The significant energy drain due to continuous expulsion of VAs results into arrest in fermentation (i.e. stuck/sluggish fermentation). An advantage of the present invention is that pH is maintained ≥pKa of these VAs as a most ideal way towards making the bioethanol fermentation process robust towards VA inhibition.

Fermentation may be performed in several steps, such as one, two, three or more steps to optimize the process, such as to allow for a continuous process rather than batch fermentation. In FIG. 1i, a three stage fermentation is shown as one embodiment of the present invention, but should not be considered as limiting for the invention.

In one embodiment, the fermentation is performed in a three stage fermentation with stage specific dilution rates to ensure specific ethanol concentrations in each stage.

In one embodiment, the fermentation is performed in three stage continuous mode, where the ethanol concentration (w/w) at the first stage is maintained between 1% to 10%, preferably with 4% to 9%, more preferably between 5% to 7% most preferably between 5.5-6.0%; such as at 5.5%, 5.6, 5.7, 5.8, 5.0, or 6.0%. The steady state concentration of ethanol in stage 1 is achieved by continuous inflow of hydrolyzed feedstock to the fermentation broth in the stage 1 fermenter and continuous outflow of fermented material from the stage 1 fermenter to the stage 2 fermenter, maintaining a dilution rate (D) in the range of 0.10-0.55 h⁻¹, preferable between 0.10-0.30 h⁻¹, more preferably between 0.10-0.20 h⁻¹. An advantage of keeping the ethanol concentration in this desired range, especially between 5.5-6.0% in the first fermenter, is that potential contaminants, such as lactic acid bacteria (LAB), acetic acid bacteria (AAB) or other will be inhibited, while yeast is not inhibited. The residence time of first fermenter is between 5 to 80 hours, preferably between 10 to 30 hours, more preferably between 12 to 15 hours.

The overflow from the first fermenter is transferred to a second fermenter where the ethanol concentration (w/w) is maintained between 6% to 18%, preferably with 8% to 16%, more preferably between 9% to 12%; such as 9%, 9.5%, 10%, 10.50%, 11%, or 12%. The steady state concentration of ethanol is achieved by continuous inflow of fermented material from the stage 1 fermenter to the stage 2 fermenter and continuous outflow of fermented material from the stage 2 fermenter to the stage 3 fermenter, maintaining a dilution rate (D) in the range of 0.04-0.10 h⁻¹, preferably between 0.04-0.08 h⁻¹, more preferably between 0.4-0.06 h⁻¹. The residence time of second fermenter is between 5 to 80 hours, between 5 to 60 hours, preferably between 10 to 40 hours, more preferably between 25 to 30 hours.

The overflow from the second fermenter is transferred to a third fermenter where the ethanol concentration (w/w) is maintained between 10% to 15%, preferably between 12% to 15%. The residence time of third fermenter is between 3 to 8 hours, preferably between 4 to 7 hours, more preferably between 5 to 6 hours. The constant ethanol concentration is achieved by continuous inflow of fermented material from the stage 2 fermenter to the stage 3 fermenter and continuous outflow of fermented material from the stage 3 fermenter to a distillation unit.

In one embodiment, yeast is added to the feedstock hydrolysate to obtain a fermentation broth. The fermentation both is fermented by continuous fermentation comprising at least two stages, preferably three stages. In one embodiment, the liquefact (feedstock hydrolysate) resulting from the enzymatic hydrolysis step is appropriately dosed to a stage 1 fermenter to maintain an ethanol concentration in the stage 1 fermenter between 5-11% (w/w); this ethanol concentration in stage 1 may be obtained by maintaining a dilution rate between 0.1 h⁻¹- to 0.55 h⁻¹ in stage 1. In a further embodiment, the fermented mash from the stage 1 fermenter is then continuously dosed to a stage 2 fermenter to maintain an ethanol concentration in the stage 2 fermenter between 10-13% (w/w); this ethanol concentration in stage 2 may be obtained by maintaining a dilution rate between 0.04 h⁻¹ to 0.1 h⁻¹. In a further embodiment, the fermented mash from the stage 2 fermenter is then dosed to a stage 3 fermenter, where ethanol concentration is maintained between 12-15% (w/w) by continuously dosing the fermented mash from stage 3 to a distillation unit.

An advantage of the continuous mode is, there is no need to stop fermentation, hence a significant saving on CIP cycles, resulting in reduced usage of water, energy as well as chemicals. Moreover decreasing CIP also results in improving overall process downtime. In ethanol production, typically around 8-10 hours are lost in cleaning and restarting batch fermentation process (downtime). In traditional ethanol plants, due to the risk of contamination, such mode of operating the process in continuous mode is avoided. The current invention overcomes this potential limitation. In one embodiment, ammonium nitrate is added during the fermentation. Not wishing to be bound by theory, it is speculated that the nitrate in ammonium nitrate is converted to ammonium ion during fermentation (nitrate respiration). The ammonium produced due to nitrate respiration keeps fermentation pH above pKa of the volatile acids. Nitrate->nitrite->ammonium. The formed ammonium is utilized by yeast as a nitrogen source (same as ammonia water). So all in all, a two level safety net is created to prevent pH going down below pKa of volatile acids: 1) by adjusting ph using ammonia water and 2) by maintaining adjusted pH by nitrate.

In one embodiment, the ammonium nitrate concentration during fermentation is between 50-500 ppm, more preferably between 100-350 ppm, most preferably between 150-250 ppm; such as at 160, 170, 190, 200, 210, 230, 250 ppm. Adding ammonium nitrate ensures maintaining pH ≥5.00 whereby the inhibitory effects from VAs on yeast are prevented.

The process of the present invention benefits from maintaining the pH around pH 5 (or higher) as this keeps the VAs in their unprotonated state whereby VA inhibition of the yeast is reduced, hence creating most favorable conditions for the yeast fermentation and ensuring less stress on the yeast, thereby avoiding potential arrest in fermentation. Further, as a result of the higher ethanol concentration obtained in the first stage of fermentation, potential contaminants are inhibited, thereby sending well adapted yeast to the second stage of fermentation, which overall lead to improved ethanol yields. Apart from this, continuous fermentation mode also results in significant reduction in downtime (time required to start up new fermentation), thus improving productivity of the entire process.

### I.vi Recovery of Ethanol

A first main product of the process of the present invention is ethanol. Ethanol may be recovered by distillation of the output from the fermentation tank. In FIG. 1, this is illustrated as “Innovation step 4” (star 4).

In one example, as recognized by a person skilled in the art, the fermented slurry may be heated to ˜60° C., such as by using a heat exchanger, and then pumped into a distillation column where the temperature may be maintained at around ˜80° C., such as by using steam generated from a stillage boiling tank (illustrated as “Innovation step 5” (star 5) in FIG. 1, further described in section I.viii). Ethanol is evaporated off, while the generated whole stillage is sent to further separation and filtration for protein recovery (illustrated as “Innovation step 6” (star 6) in FIG. 1B, further described in section I.vii).

Evaporated ethanol may further be rectified to achieve ˜₉₅% ethanol concentration. A molecular sieves may further be applied to dehydrate the ethanol product to a concentration of 99.99%. At the end, the dehydrated ethanol may be denatured by adding an appropriate denaturant.

### I.vii Recovery of Protein

A second main product of the process of the present invention is a protein feed or food product. In one abonnement, the whole stillage from the distillation process is processed to produce a high protein product, such as a protein product comprising at least 40, 45, 50, 55, 60, 65, 70, 75%, or at least 80% protein in the final composition. Protein products having high protein content (w/w) are highly desirable.

To obtain such high protein product, the whole stillage must undergo separation and/or purification steps. In FIG. 1, this is illustrated as “Innovation step 6 and 7” (star 6 and 7).

In one embodiment, the whole stillage is separated in two fractions: fiber and filtrate. Separation may be achieved by using existing methodologies, such as decanter, centrifuge, filter press, pressure screen, and hydrocyclone, either alone or in combination. In one example, whole stillage is passed through a pressure screens (such as a screen having a 75 μm sieve size). The resulting de-fibered filtrate may further be separated into high density and low density slurry fractions. Separation can be achieved by using either of existing methodologies, such as decanter, centrifuge, filter press, pressure screen, hydrocyclone, either alone or in combination. In one example, the de-fibered filtrate is passed through a hydro-cyclone resulting in two streams: a high density stream with insoluble solids (˜40% dry matter) and a low density overflow thin stillage stream containing soluble solids (˜5% dry matter). The high density stream may further be dehydrated in a spray drier column using hot air (˜150° C.), resulting in a high protein powder product. In one embodiment, the protein recovered in the process is spray-dried.

An advantage of the process is that the final protein product comprises none or only very little fiber content. Meanwhile, the separate fiber side-stream can be sold as co-product or further valorized to a high value product (such as soluble dietary fibers).

In one embodiment, the whole stillage is separated into two fractions: thin stillage containing soluble fibers, fat, salts and proteins and wet cake comprising of insoluble fiber and protein. The oil or fat content of thin stillage is further removed using separation technologies like disc stack centrifuge or decanter. The defatted or de oiled thin stillage is boiled (step I.viii) and recycled back. The wet cake is dried using conventional driers like tray, ring or DDGS drier to obtain high protein.

In one embodiment, the oil or fat content in the wet cake is removed using organic solvents like supercritical CO₂ or hexane or octanol.

I.viii Boiling Low Density Stillage

In one embodiment, low density stillage derived from the protein recovery process (see section I.vii) is boiled using existing technologies, such as by natural gas fired boiler, energy recuperation from jet cooker, or other process steps. In FIG. 1, this is illustrated as “Innovation step 5” (star 5).

As an example, low density stillage is boiled using jacket heating where steam from boiler is used. The generated steam vapors from the low density stillage are used to distill ethanol (refer to section I.vi Recovery of ethanol). This low density stillage comprises volatile acids (originating from the liquefaction (cold mashing) stage, where backset is mixed with milled feedstock to make slurry). During the boiling process these VAs are evaporated, and the resulting backset stream with a low VA concentration (≤0.2%) is (re)used to mix with the feedstock in the initial steps of the overall process (refer to section I.iii Hydolysis). This use of backset water from the boiling of the low density stillage reduces the overall fresh water consumption of the process, which is illustrated in Example 7, where fresh water consumption is found to be reduced from 2.8 l water per l. ethanol to 0.6 l. water per l. ethanol. The reduced concentration of VAs in the backset ensures that there is no negative impact on the ethanol yield by using this backset water. Boiling of the low density stillage also reduces the risk of contamination originating from thin stillage. Further, boiling of the low density stillage ensures up-concentration of soluble proteins and other moieties present in the stillage since steam (water) is lost from the stillage; also increased dry substance in backset due to evaporation of the thin stillage.

### I.ix Recovery of CO₂

A third main product of the process of the present invention is CO₂. In FIG. 1, this is illustrated as “Innovation step 4” (star 4). In one embodiment, CO₂ is captured by dosing carbonic anhydrase in the fermentation process. Carbonic anhydrase is an enzyme that assists rapid inter-conversion of carbon dioxide and water into carbonic acid, protons and bicarbonate ions. The carbonic anhydrase enzyme belongs to the E.C. 4.2.1.1 enzyme classification. One example of such carbonic anhydrase enzyme is carbonic anhydrase from bovine erythrocytes sold by Merck cas no. 9001-03-0.

Carbonic anhydrase may be added to one of more of the fermentation tanks. In a preferred embodiment, the carbonic anhydrase is added to all the fermentation tanks (innovation step 3 (star 3) in FIG. 1). As disclosed previously, the fermentation may be performed in multiple steps. In one preferred embodiment, the carbonic anhydrase is added in fermentation tank 1, 2 and 3 (numbers referring to the first, second and third fermenter mentioned in section Iv), more preferably carbonic anhydrase is added in the fermentation tank 1 and 2. In a further embodiment, carbonic anhydrase is added only in the first fermentation tank, but will be carried over to the next tank(s) as the fermentation broth is moved to the next tank as part of the continuous operation

In one embodiment, CO₂ is captured during the fermentation by addition of carbonic anhydrase to the fermentation broth, such as preferably directly added to the feedstock hydrolysate together with addition of yeast, or some time after. In one preferred embodiment, ZnSO4 is additionally added to the fermentation broth. The term fermentation broth refers to the feed stock slurry in a fermenter, which depending on the progression of the fermentation process may comprise the initial feedstock hydrolysate in combination with the fermented feedstock broth.

In one embodiment, the amount of carbonic anhydrase enzyme added to the fermentation is 0.1-2 kg/ton dry matter, preferably 0.1-1.5 kg/ton dry matter, more preferably 0.2-1 kg/ton dry matter, most preferably 0.3-0.5 kg/ton dry matter.

In one embodiment, the amount of carbonic anhydrase enzyme added to the fermentation is 200-4000 Units/gram dry matter, preferably 200-3000 Units/gram dry matter, more preferably 400-2000 Units/gram dry matter, most preferably 600-1000 Units/gram dry matter. The enzyme units refer to Wilbur-Anderson Units (W-A units). One W-A unit will cause the pH of a 0.02 M Trizma buffer to drop from 8.3 to 6.3 per minute at zero degree Celsius.

In one embodiment zinc sulphate is added to the fermentation broth to ensure an amount of 0.6 to 6.0 mmol Zn⁺ per kg of carbonic anhydrase in the fermentation broth. In one embodiment the amount of Zn⁺ in the fermentation broth is 0.6 to 6.0 mmol Zn⁺ per kg of carbonic anhydrase, 0.6 to 4.8 mmol Zn⁺ per kg of carbonic anhydrase, 0.6 to 3 mmol Zn⁺ per kg of carbonic anhydrase, 1.2 to 2.4 mmol Zn⁺ per kg of carbonic anhydrase, or approximately 1.8 mmol Zn⁺ per kg of carbonic anhydrase.

In one embodiment zinc sulphate is added to the fermentation broth to ensure an amount of 0.3×10⁻⁹ to 3.0×10⁻⁹ mmol Zn⁺ per unit of carbonic anhydrase in the fermentation broth. In one embodiment the amount of Zn⁺ in the fermentation broth is 0.3×10⁻⁹ to 3.0×10⁻⁹ mmol Zn⁺ per unit of carbonic anhydrase, 0.3×10⁻⁹ to 2.4×10⁻⁹ mmol Zn⁺ per unit of carbonic anhydrase, 0.3×10⁻⁹ to 1.5×10⁻⁹ mmol Zn⁺ per unit of carbonic anhydrase, 0.6×10⁻⁹ to 1.2×10⁻⁹ mmol Zn⁺ per unit of carbonic anhydrase, or approximately 0.9×10⁻⁹ mmol Zn⁺ per unit of carbonic anhydrase.

In one embodiment zinc sulphate is added to the fermentation broth to ensure a concentration of 0.1 to 1.0 gram ZnSO₄ per 100 gram of carbonic anhydrase in the fermentation broth. In one embodiment the concentration of ZnSO₄ in the fermentation broth is 0.1 to 1.0 gram ZnSO₄ per 100 gram of carbonic anhydrase, 0.1 to 0.8 gram ZnSO₄ per 100 gram of carbonic anhydrase 0.1 to 0.5 gram ZnSO₄ per 100 gram of carbonic anhydrase, 0.2 to 0.4 gram ZnSO₄ per 100 gram of carbonic anhydrase, or approximately 0.3 gram ZnSO₄ per 100 gram of carbonic anhydrase. In one embodiment, the amount of ZnSO₄ in the fermentation broth is 0.1, 0.2 0.3, 0.4, 0.5, 0.6, 0.7, 0.8, 0.9, or 1.0 g/100 g carbonic anhydrase.

In one embodiment zinc sulphate is added to the fermentation broth to ensure a concentration of 0.05×10⁻⁸ to 0.5×10⁻⁸ gram ZnSO₄ per unit of carbonic anhydrase in the fermentation broth. In one embodiment the concentration of ZnSO₄ in the fermentation broth is 0.05×10⁻⁸ to 0.5×10⁻⁸ gram ZnSO₄ per unit of carbonic anhydrase, 0.05×10⁻⁸ to 0.4×10⁻⁸ gram ZnSO₄ per unit of carbonic anhydrase 0.05×10⁻⁸ to 0.25×10⁻⁸ gram ZnSO₄ per unit of carbonic anhydrase, 0.1×10⁻⁸ to 0.2×10⁻⁸ gram ZnSO₄ per unit of carbonic anhydrase, or approximately 0.15×10⁻⁸ gram ZnSO₄ per unit of carbonic anhydrase. In one embodiment, the amount of ZnSO₄ in the fermentation broth is 0.05×10⁻⁸, 0.1×10⁻⁸, 0.15×10⁻⁸, 0.2×10⁻⁸, 0.25×10⁻⁸, 0.3×10⁻⁸, 0.35×10⁻⁸, 0.4×10⁻⁸, 0.45×10⁻⁸, 0.5×10⁻⁸ g/unit carbonic anhydrase.

In one preferred embodiment, Zn+ is added together with the carbonic anhydrase, where the amount of carbonic anhydrase added to the fermentation is 0.3-0.5 kg/ton of dry matter, and the amount of Zn+ is 1.2-2.4 mmol/kg carbonic anhydrase, wherein the enzyme activity is 2000 units/mg enzyme.

The use of carbonic anhydrase for CO₂ capture and use of heating to release the captured CO₂ is illustrated in Example 5.

The carbonic acid formed from capturing CO₂ (conversion of CO₂ by carbonic anhydrase) lowers the pH of the fermentation media, hence triggering dissociation of the carbonic acid back to CO₂ and water. However, due to continuous pH adjustment, such as by using ammonia water as described in section Liv and Iv, CO₂ remains trapped in the form of bicarbonate (HCO₃⁻), thus avoiding its release during fermentation. In one embodiment, the continuous pH adjustment as described above is done in the first stage of fermentation. In one embodiment, the continuous pH adjustment is done only in the first stage of fermentation, not in any subsequent fermentation stages. Specifically, the continuous pH adjustment is performed to ensure pH is between 5.5-6.5 in the fermentation.

The captured CO₂ is released in the distillation stage. The capture of CO₂ results in lowering the ethanol concentration in the fermentation. As CO₂ is not lost, the fermentation volume will remain more or less constant during the fermentation, thus reducing ethanol concentration. Ethanol above 6% concentration induces stress in yeast. The stress increases as ethanol concentration increases. In the present process, the ethanol induced stress is reduced, leading to improved conditions for the yeast, which leads to a smooth fermentation. Further, due to minimal CO₂ loss, evaporation also reduces significantly, resulting in more water in the fermenter, leading to diluting ethanol concentration. Due to continuous mode, ethanol concentration remains ˜10%, thus reducing yeast stress.

Further, capture of CO₂ results in lowering ethanol loss due to evaporation. Around 0.5% ethanol is lost in evaporation. In ethanol fermentation, approximately 40% of ethanol is present in vapor form, some of this ethanol is lost as a result of CO₂ draft (CO₂ escaping).

Typical ethanol plants employ “primary scrubber system” on the vent coupled to the fermentation vessels and other process gas discharges to minimize the discharge of vaporous ethanol and other volatile organic compounds (“VOCs”) from escaping into the atmosphere with the carbon dioxide and other vapors. As appreciated by those skilled in the art, VOCs may include compounds such as acetaldehyde, ethyl acetate, acrolein, and acetone. These scrubbers use fresh water along with a variety of additives to increase the solubility of the ethanol and VOCs. The precipitate of the scrubber is water with a low concentration of ethanol. This water mixture is then reintroduced into the process as make-up water which is then taken through the heating cycles along with the mash. It is in this area that the temperature is often taken over the flash point of ethanol, which in turn allows the ethanol to be lost to a vent system and destroyed in a thermal oxidizer. Consequently, both ethanol and VOCs are discharged into the atmosphere which reduces the ethanol yield and pollutes the environment.

In one embodiment of the present invention, fermenter scrubbers are not needed, thereby resulting in preventing ethanol loss, significant reduction in freshwater consumption, avoiding addition of additives, and reducing energy consumption by avoiding heating cycles.

To trap and release CO₂, typical ethanol plants use “absorption-desorption columns”. The washed and clean CO₂ from the primary scrubber system is trapped into absorption column by solvents like monoethanolamine (MEA). The CO₂-MEA complex passes through “desorption” column where temperature is increased to ˜120° C. where CO₂ is released, and MEA is regenerated and recycled back to absorption column.

In one embodiment of present invention, the MEA based absorption/desorption system is not needed, which is an advantage as the use of chemicals like MEA is avoided and energy consumption is reduced by avoiding the desorption step.

In one embodiment, the CO₂ produced and captured during the fermentation may be released in the form of CO₂ vapors in a distillation step. In one embodiment, both the CO₂ captured during fermentation as well as the ethanol produced during fermentation are vaporized by the application of heat in the distillation step, and separated using a condenser, where the ethanol is condensed in liquid form and the CO₂ remains in gaseous form, hence the CO₂ may be recovered separately from the ethanol.

In one illustrative embodiment, the distillation temperature for vaporization of the CO₂ and ethanol is around 70-90° C., preferably around 80° C., and the condensation temp is around 5-30° C., preferably around 5° C. Finally, the released CO₂ from the condenser step may further be purified using traditional compression-expansion systems.

In one embodiment, the process comprises three, two or one compression-expansion cycles; in a preferred embodiment the purification process comprises only one compression-expansion cycle, resulting in significant reduction in energy requirements.

### I.x Reuse of Process Water

One important aspect of the invention is the reduced need/use of fresh water, compared to a traditional ethanol production process. Several of the process steps of the present invention contribute to this significantly reduced water need.

In one embodiment, the water consumption is reduced by 50, 55, 60, 65, 70, 75% or even by 80% compared to state of the art ethanol production process (FIG. 1A vs FIG. 1). An illustrative example is provided in Example 7, showing calculations for reduced water consumption.

For example, the water contribution from feedstock material is significantly increased, such as increased by 50% (see Example 7), as a result of not needing to dry the feedstock material.

Further, the reuse of process water within the process contributes significantly to the overall reduction of water consumption. The process water may be reused anywhere relevant for the process. An example of reuse of process water is provided in Example 2.

The process water for reuse in the process may or may not additionally undergo purification step(s) to remove impurities prior to its reuse. Preferably, the process water does not undergo any purification steps, such as chemical or physical water purification process, before being reused in the process. The microbial pretreatment discussed in section I.ii helps ensure the process water is clean and free of unwanted contaminant which in themselves are undesirable, but which may also produce products such as VAs which are undesirable to have in the process water.

The process of the present invention significantly reduces the amount of process water categorized as waste. As illustrated in FIG. 1, the process water is reused for the conditioning of the feedstock. before the hydrolysis step.

In one embodiment, condensate water is returned to the boiler—illustrated as “innovation step 5 (star 5) in FIG. 1B—resulting in reducing water consumption by 0.6 l. water per l. of ethanol produced.

In one embodiment, the method further comprises recovering of aqueous liquid from one or more process steps of the method. This aqueous liquid is also herein referred to as process water. In one preferred embodiment, the aqueous liquid recovered from the process is reused within the process by adding it to the feedstock (to obtain the feedstock slurry for subsequent hydrolysis as disclosed in section I.iii). Hence, in one embodiment the aqueous liquid added to the feedstock comprises the recovered process water. In one embodiment, more than 10%, 15%, 20%, 25%, 30%, 35%, 40%, 45%, or even more than 50% of the aqueous liquid added to the feedstock is process water recovered from one or more other steps of the method.

In one embodiment, process water is recovered from the distillation step and this recovered water is reused in other process steps of the method, as disclosed herein.

### II. Products of the Present Invention

### I.i Ethanol

One of the products obtained from the present method is ethanol. As discussed above, the ethanol yields are improved compared to traditional ethanol processes.

In one embodiment, the ethanol purity is at least 99.3%; Density at 20 degrees Celsius is maximum 0.7915 kg/liter; Water content is maximum 0.5% (by mass); Methanol is maximum 0.5% (by mass), and total acids (as acetic acid) is maximum 0.007% (by mass)

### II.ii Protein Feed or Food Product

One of the products obtained from the present method is protein.

The term “protein feed product” refers to a protein product which based on its purity can be used as feed, such as for animal consumption.

The term “protein food product” refers to a protein product which based on its purity can be used as food, such as for human consumption.

In the present context, purity is measured as amount of toxins—such as deoxynivalenol (DON), 3-acetyldeoxynivalenol (3-ADON), 15-acetyldeoxynivalenol (15-ADON), nivalenol (NIV), and zearalenone (ZON). By applying probiotics to the feedstock material as an initial step of the present invention, the protein product obtained by the method of the present invention is essentially pure in the sense that it is free of toxins, as no other potentially toxic microbial contaminations can arise in the feedstock material due to the presence of the probiotic microbes. The probiotic microbes themselves do not produce toxins.

Fungal contamination is the biggest challenge in ethanol fermentation due to its toxins. For a protein product, which goes into human food or animal feed, having fungal toxins present, such as aflatoxins, are highly unfavorable and also not allowed in many jurisdictions. However, the presence of lactic acid bacteria (dead cells, as everything will be killed on distillation column) is on the contrary seen as added value, as it is non gmo probiotic.

In one embodiment, the protein product has Dry matter of 90%; Crude protein of ≥60%; Crude fat of ≥8%; and Essential amino acid (for example, Lysine) of ≥2%.

### II.iii CO₂

One of the products obtained from the present method is CO₂.

In one embodiment, the Purity of CO₂ is 99.9%; Moisture is maximum 20 ppmv; Oxygen is maximum 30 ppmv; Carbon monoxide is maximum 10 ppmv; Ammonia is maximum 2.5 ppmv; Methanol is maximum 10 ppmv; Acetaldehyde is maximum 0.2 ppmv; Sulfur dioxide (SO2) is 1 ppmv; No foreign Odor of solid CO₂ (snow); No foreign appearance of solid CO₂ (snow); No foreign odor or taste in water; No color or turbidity in water.

### III. Advantages and Commercial Application

As disclosed above, the method of the present invention provides many advantages compared to traditional ethanol fermentation and has direct commercial application. The following list summarizes some of the advantages of the present process:

Reducing water consumption in the ethanol process. In example 7, it is calculated that water consumption is reduced from 2.8 l. fresh water per l. of ethanol in a traditional ethanol process, to 0.6-l. fresh water per l. of ethanol for the present process.

Reducing CIP cycles from 120 cycles per year to 40, resulting in downtime reduction of ˜400 hours/year of CIP related activities (each CIP cycle is ˜5 hours).

Producing a pure high protein product is produced which may be used as food or feed.

### Reducing Energy Requirement

Reducing GHG emissions on ethanol.

Improving productivity of ethanol fermentation by eliminating or reducing downtime.

### Eliminating Grain Drying Step

Improving microbial stability of waste grain and grain material enabling better upcycling.

Energy efficient CO₂ capture.

### Preferred Numbered Embodiments of the Invention

Numbered embodiment 1. A method for making ethanol and a protein feed or food product, from grain and/or grain-derived products, comprising the steps of:
providing grain and/or grain-derived products,
pretreating the grain and/or grain-derived products by applying a probiotic microorganism to the grain and/or grain-derived products,
adding an aqueous liquid to the pretreated grain and/or grain-derived products, followed by mixing to obtain a grain slurry,
optionally adjusting pH of the grain slurry to ≥5.5 by addition of ammonia water to the grain slurry,
enzymatically treating the grain slurry by addition of an amylase enzyme to the grain slurry to obtain a grain hydrolysate,
fermenting the grain hydrolysate by addition of yeast to the grain hydrolysate, and
separately recovering
ethanol
protein, and
optionally CO₂

Numbered embodiment 2. The method according to numbered embodiment 1, wherein the probiotic microorganism in step (b) is applied onto the surface of the grain and/or gran-derived product, such as by spraying the microorganism on the surface.

Numbered embodiment 3. The method according to numbered embodiment 1 or 2, wherein step (g) further comprises recovering process water; and wherein the aqueous liquid added to the grain and/or grain-derived products in step (c) comprises the recovered process water.

Numbered embodiment 4. The method according to any one of numbered embodiments 1-3, wherein the grain in step (a) is selected from wheat, rice, oats, barley, rye, barley, millet, corn, triticale, and sorghum grain.

Numbered embodiment 5. The method according to any one of numbered embodiments 1-4, wherein the probiotic microorganism in step (b) is a lactic acid bacteria, such as selected from Lactobacillus reuteri, Lactobacillus brevis, Lactobacillus plantarum, Lactobacillus amylovorus, and Lactobacillus hammesii.

Numbered embodiment 6. The method according to any one of numbered embodiments 1-5, wherein the ammonia water in step (d) is derived from a biogas plant waste stream.

Numbered embodiment 7. The method according to any one of numbered embodiments 1-6, wherein the enzymatic treatment in step (e) is performed at a temperatures within the range of 50-55° C.

Numbered embodiment 8. The method according to any one of numbered embodiments 1-7, wherein the ethanol in step (g) is recovered by distillation.

Numbered embodiment 9. The method according to any one of numbered embodiments 1-8, wherein the protein recovered instep (g) is spray-dried.

Numbered embodiment 10. The method according to any of numbered embodiments 1-9, wherein the CO₂ in step (g) is recovered by use of carbonic anhydrase.

### EXAMPLES

### Example 1A: Pretreatment of grain material with probiotic bacteria: Lactobacillus plantarum 1A7 & W. anomalus LCF1694

Lab scale microbial stability on bread was done using Lactobacillus plantarum 1A7. Lactobacillus plantarum JA7 was propagated for 24 h at 30° C. in MRS (non-selective medium for growth of lactic acid bacteria, Oxoid Laboratories, Hampshire, United Kingdom) with the addition of fresh yeast extract (5% [vol/vol]) and 28 mM maltose to a final pH of 5.6 (mMRS). W. anomalus LCF1694 propagated for 48 h at 30° C. in YEPG (10 g/liter yeast extract, 10 g/liter peptone, 20 g/liter glucose). The antifungal activities of Lactobacillus plantarum 1A7 & W. anomalus LCF1694 was checked by in vitro challenge study to control the spoilage of bread samples caused by Mucor sp. VBBM7 and Aspergillus fumigatus MTCC2796. Bread samples were cut into small pieces (4.0+0.3 cm/8.2+0.4 cm), sterilized in petriplates and were divided into six sets. The first and second set was treated only with Lactobacillus plantarum JA7 and W. anomalus LCF1694 cell suspension respectively. The third and the fourth sets were separately inoculated with Mucor sp. (3.7×10⁴ spores/ml) and Aspergillus fumigatus MTCC2796 (4.6×10⁴ spores/ml) respectively. In the fifth set, Lactobacillus plantarum 1A7 & W. anomalus LCF1694 cell suspension was added before treatment with Mucor and Aspergillus sp. A sixth set was kept where bread pieces were not treated with any organisms. Same procedure (sterilization & inoculations) was repeated with grains from wheat, corn, triticale & rice. Care was taken to adjust grain moisture to ˜30% by sterile water.

All the samples were kept at room temperature (26.0+2.0° C.) up to 30 days and incidence of spoilages were observed.

TABLE 1A
Microbial stability of feedstock

| Set | After 2 days | After 4 days | After 6 days | After 8 days |
|---|---|---|---|---|
| Set 1 | Mold | Mold | Mold | Mold |
| | contamination | contamination | contamination | contamination |
| | absent | absent | absent | absent |
| Set 2 | Mold | Mold | Mold | Mold |
| | contamination | contamination | contamination | contamination |
| | absent | absent | absent | absent |
| Set 3 | Mold | Mold | Mold | Mold |
| | contamination | contamination | contamination | contamination |
| Set 4 | Mold | Mold | Mold | Mold |
| | contamination | contamination | contamination | contamination |
| Set 5 | Mold | Mold | Mold | Slight Mold |
| | contamination | contamination | contamination | contamination |
| | absent | absent | absent | seen |
| Set 6 | Contamination | Contamination | Contamination | Contamination |

The bread treated with probiotics remained microbially stable even after challenge test where spores from Aspergillus fumigatus and Mucor was sprinkled over probiotic treated bread. The controls challenged with Aspergillus fumigatus or Mucor, not having received any probiotic treatment, showed massive growth of the Aspergillus fumigatus and Mucor. Interestingly even the control which had not been treated with probiotics nor been challenged with Aspergillus fumigatus or Mucor also got contaminated in seven days as seen from the depression in the bread.

Conclusion: Lactobacillus plantarum 1A7 and W. anomalus LCF1694 prevented mold contamination in grain as well as in bread for almost one week.

### Example 1B: Pretreatment of Bread with Probiotic Bacteria: Bacillus Species

Lab scale microbial stability on bread made from wheat flour was done using bacillus species; Bacillus licheniformis, Bacillus Clausii, Bacillus Subtilis, Bacillus amyloliquefaciens, Bacillus fusiformis and Bacillus megaterium.

All bacillus cultures were maintained on 3% trypticase soy broth (TSB) and 0.5% yeast extract agar plates were inoculated into test tubes with 3.5 mL of 3% TSB and 0.5% yeast extract. Following 24 h of incubation at 37° C. the cultures were transferred to 1 or 2 L volumes of 0.3% TSB in a 1 tube to 1 L ratio. After six days of incubation at 37° C., spores are harvested. The antifungal activities of bacillus species were checked by in vitro challenge study to control the spoilage of bread samples caused by Mucor sp. VBBM7 and Aspergillus fumigatus MTCC2796. Bread samples were cut into small pieces (4.0±0.3 cm/8.2±0.4 cm), sterilized in Petri plates and were divided into nine sets.

The first set was treated only with bacillus species cell suspension. The second and the third sets were separately inoculated with Mucor sp. (3.7×10⁴ spores/ml) and Aspergillus fumigatus MTCC2796 (4.6×10⁴ spores/mi), respectively. In the fourth set, Bacillus licheniformis cell suspension was added before treatment with Mucor and Aspergillus sp. In the fifth set, Bacillus clausii cell suspension was added before treatment. In the sixth set, Bacillus amyloliquefaciens cell suspension was added before treatment. In the seventh set, Bacillus subtilis cell suspension was added before treatment. In the seventh set, Bacillus megaterium cell suspension was added before treatment. Finally in eighth set a suspension was made using all the bacilli species and was added before treatment with Mucor and Aspergillus sp. A ninth set was kept where bread pieces were not treated with any organisms.

Same procedure (sterilization & inoculations) was repeated with grains from wheat, corn, triticale & rice. Care was taken to adjust grain moisture to 300 by sterile water.

All the samples were kept at room temperature (30.0+2.0° C.) up to 30 days and incidence of spoilages were observed.

TABLE 1B
Microbial stability of bread (grain derived product)

| Set | After 2 days | After 4 days | After 6 days | After 8 days |
|---|---|---|---|---|
| Set 1 | Mold | Mold | Mold | Mold |
| | contamination | contamination | contamination | contamination |
| | absent | absent | absent | absent |
| Set 2 | Mold | Mold | Mold | Mold |
| | contamination | contamination | contamination | contamination |
| Set 3 | Mold | Mold | Mold | Mold |
| | contamination | contamination | contamination | contamination |
| Set 4 | Mold | Mold | Mold | Slight Mold |
| | contamination | contamination | contamination | contamination |
| | absent | absent | absent | seen |
| Set 5 | Mold | Mold | Slight Mold | Slight Mold |
| | contamination | contamination | contamination | contamination |
| | absent | absent | seen | |
| Set 6 | Mold | Mold | Slight Mold | Mold |
| | contamination | contamination | contamination | contamination |
| | absent | absent | seen | |
| Set 7 | Mold | Mold | Mold | Slight mold |
| | contamination | contamination | contamination | contamination |
| | absent | absent | absent | seen |
| Set 8 | Mold | Mold | Slight mold | Mold |
| | contamination | contamination | contamination | contamination |
| | absent | absent | absent | absent |
| Set 9 | Mold | Mold | Mold | Mold |
| | contamination | contamination | contamination | contamination |
| | absent | absent | absent | absent |

Conclusion: Most of the bacillus species can control growth of mold up to 4-6 days. Interestingly consortia of bacilli can prevent mold contamination for more than 8 days.

### Example 2: Reuse of Process Water and pH Adjustment Using Ammonia Water

Lab scale simulation of untreated backset containing volatile acids ≥1 g/l to milled flour was carried out using shake flasks (75 g starting weight) where white bread was milled and mixed with water to achieve 30% slurry dry matter. Acetic and Lactic acid solutions were added in shake flask 3, 4, 5 and 6 to achieve 1% and 0.5% concentration respectively. pH was adjusted to 6.20 in flask no 5 and 6 using 20% ammonia solution. Alpha amylase, Novozymes thermostable alpha amylase Liquoflow GO 2× (Dose; 0.2 kg/ton bread) was added and liquefaction was carried out at 85° C. for 2 hours. Please note that optimal temperature range for this thermostable alpha amylase is between 75-86° C. The resultant liquefied mash was cooled down to 32° C. Appropriate quantity of Urea was added in all the shake flasks to achieve 250 ppm inorganic nitrogen concentration. Around 1 gm of dry yeast Innova Fit from Novozymes was added in all the shake flasks. Finally Novozymes glucoamylase containing glucoamylase & cellulose, Saczyme yield (Dose; 0.5 kg/ton bread) was added in all the flasks. Please note that this enzyme is different than alpha amylase used in the liquefaction. The main purpose of this enzyme is to generate glucose. Moreover, the optimal temperature range for this enzyme is between 30 −40° C. All the flasks were kept in incubator shaker (32° C. at 80 RPM). After 72 hours, sample was taken from the flasks and key parameters like pHh, glucose, ethanol was measured.

TABLE 2
Effect of adjusting pH ≥ 5.50

| | % | % lactic | | | | |
|---|---|---|---|---|---|---|
| | Acetic | acid at | pH | | Ethanol | Glucose |
| | acid at | the start | adjustment | pH at | at the | at the |
| | the start | of | to 6.20 | the end | end of | end of |
| Shake | of | fermen- | using 20% | of the | fermen- | fermen- |
| flask | fermen- | tation | ammonia | fermen- | tation | tation |
| no | tation | (%) | solution | tation | (%) | (%) |
| 1 | 0 | 0 | No, as is, 5.12 | 4.50 | 9.41 | 0.08 |
| 2 | 0 | 0 | No, as is, 5.08 | 4.52 | 9.29 | 0.06 |
| 3 | 1 | 0.5 | No, 3.90 | 3.30 | 0.98 | 16.5 |
| 4 | 1 | 0.5 | No, 3.85 | 3.27 | 0.72 | 17.1 |
| 5 | 1 | 0.5 | Yes, 6.20 | 5.21 | 9.25 | 0.05 |
| 6 | 1 | 0.5 | Yes, 6.20 | 5.25 | 9.39 | 0.07 |

As seen from the above-mentioned results, it is confirmed that pH adjustment to 6.20 by ammonia water prevents acetic and lactic acid inhibition without affecting ethanol titers.

### Example 3: Raw Starch Hydrolysis (Cold Mashing) Process

Lab scale experiments was carried out using shake flasks (75 g starting weight) where white bread was milled and mixed with water to achieve 3000 slurry dry matter. pH was measured (but not adjusted). Liquoflow GO 2× (thermostable alpha-amylase, Dose; 0.2 kg/ton bread) was added in shake flask 1 and 2, and liquefaction was carried out at 85° C. for 2 hours.

Raw starch hydrolysis enzyme from Novozymes, Saczyme cold mash comprise of glucoamylase, acid amylase and cellulose with dose; 1 kg/ton bread was added in shake flask 3 and 4, and liquefaction was carried out at 45° C. for 30 min. The resultant liquefied mash was cooled down to 32° C. Appropriate quantity of Urea was added in all the shake flasks to achieve 250 ppm inorganic nitrogen concentration. Around 1 gm of dry yeast (Innova Fit) was added in all the shake flasks. Finally, gluco amylase, Saczyme yield (Dose; 0.5 kg/ton bread) was added in all the flasks There is some gluco amylase activity in Saczyme cold mash, the reason to add another gluco amylase, Saczyme yield, later in the fermentation is to boost saccharification process. All the flasks were kept in incubator shaker (32° C. at 80 RPM). After 72 hours, sample was taken from the flasks and key parameters like pH, glucose, ethanol was measured.

TABLE 3
Ethanol titers at the end of fermentation
in baseline and cold mashing process

| Shake | Ethanol at the end | Glucose at the end |
|---|---|---|
| flask no | of fermentation (%) | of fermentation (%) |
| 1 | 9.41 | 0.08 |
| 2 | 9.29 | 0.06 |
| 3 | 9.18 | 0.09 |
| 4 | 9.39 | 0.10 |

As seen from the above mentioned results, it is confirmed that similar ethanol titers can be achieved using cold mashing process.

### Example 4: Ammonium Nitrate as Inorganic Nitrogen Source

Lab scale experiment was carried out using shake flasks (75 g starting weight) where white bread was milled and mixed with water to achieve 30% slurry dry matter. pH was measured (but not adjusted). Raw starch hydrolysis enzyme, Saczyme cold mash was added (Dose; 1 kg/ton bread) in all the shake flasks and cold liquefaction (also called raw starch hydrolysis) was carried out at 45° C. for 30 min. The resultant liquefied mash's were cooled down to 32° C. Appropriate quantity of Urea was added in the shake flask 1 and 2 to achieve 250 ppm inorganic nitrogen concentration. Appropriate quantity of Ammonium Nitrate was added in shake flask 3 and 4 to achieve 250 ppm inorganic nitrogen concentration. Around 1 gm of dry yeast (Innova Fit) was added in all the shake flasks. Finally, amino glucosidase, Saczyme yield (Dose; 0.5 kg/ton bread) was added in all the flasks. All the flasks were kept in incubator shaker (32° C. at 80 RPM). After 72 hours, sample was taken from the flasks and key parameters like pH, glucose, ethanol was measured.

TABLE 4
Effect of inorganic nitrogen source and
ethanol titers at the end of fermentation

| | | | Ethanol at | Glucose at |
|---|---|---|---|---|
| Shake | pH at the | pH at the | the end of | the end of |
| flask | start of the | end of the | fermentation | fermentation |
| no | fermentation | fermentation | (%) | (%) |
| 1 | 5.20 | 4.20 | 9.21 | 0.11 |
| 2 | 5.20 | 4.15 | 9.29 | 0.09 |
| 3 | 5.19 | 5.10 | 9.18 | 0.14 |
| 4 | 5.20 | 5.05 | 9.39 | 0.08 |

As seen from the above mentioned results, it is confirmed that pH was maintained around starting pH using ammonium nitrate as inorganic nitrogen source.

It was observed that pH did not drop below 5 in the batches containing ammonium nitrate. It is possible that besides nitrate respiration, deamination might also be one of the reasons behind the same.

### Example 5A: Carbonic Anhydrase to Capture CO₂

Lab scale experiment was carried out using shake flasks (75 g starting weight) where white bread was milled and mixed with water to achieve 30% slurry dry matter. Raw starch hydrolysis enzyme, Saczyme cold mash was added (Dose; 1 kg/ton bread) in all the shake flasks and liquefaction was carried out at 45° C. for 30 min. The resultant liquefied mash was cooled down to 32° C. Appropriate quantity of Urea was added in all the shake flask to achieve 250 ppm inorganic nitrogen concentration. Around 1 gm of dry yeast (Innova Fit) was added in all the shake flasks. Amino glucosidase, Saczyme yield (Dose; 0.5 kg/ton bread) was added in all the flasks. Carbonic anhydrase (Dose: 0.3 kg/ton bread) was added in flask no 3 to 6. Finally, ZnSO₄ was added in flask 5 & 6 ensuring ˜0.3 gram ZnSO₄/100 gram Carbonic anhydrase enzyme. All the flasks were kept in incubator shaker (32° C. at 80 RPM). pH was measured twice a day in all the flasks. In flask 3-6, pH is adjusted to ˜5.5-6.0 using ammonia water. In all the flasks, CO₂ concentration was monitored qualitatively by adding few ml of CO₂ indicator fluid (Aqua Rebell CO₂ Check, bought from local aquarium shop) in the trap and color was monitored twice a day. After 72 hours, sample was taken from the flasks and key parameters like weight loss (escaped CO₂), pH, glucose, and ethanol was measured.

TABLE 5A
Effect if Carbonic anhydrase

| | | | | | | | Qualitative |
|---|---|---|---|---|---|---|---|
| | pH at | | | Ethanol | Glucose | | CO2 |
| | the | pH at | | at the | at the | | capture |
| | start | the end | | end of | end of | CO2 | based on |
| Shake | of the | of the | Weight | fermen- | fermen- | check | CO2 check |
| flask | fermen- | fermen- | loss | tation | tation | reagent | reagent |
| no | tation | tation | (g) | (%) | (%) | color | color* |
| 1 | 5.20 | 4.20 | 10.0 | 9.18 | 0.08 | Yellow | − |
| 2 | 5.20 | 4.15 | 9.50 | 9.12 | 0.05 | Yellow | − |
| 3 | 5.20 | ~5.20 | 1.82 | 8.30 | 0.03 | Bluish | + |
| | | | | | | green | |
| 4 | 5.19 | ~5.20 | 2.10 | 8.41 | 0.02 | Bluish | + |
| | | | | | | green | |
| 5 | 5.20 | ~5.20 | 1.20 | 8.0 | 0.02 | Blue | ++ |
| 6 | 5.10 | ~5.20 | 1.15 | 8.12 | 0.01 | Blue | ++ |

*− indicates no CO₂ capture in the fermentation broth,
+ indicated CO₂ captured in the fermentation broth

As seen from the above mentioned results, it is confirmed that carbonic anhydrase captures CO₂ and prevents CO₂ loss/escape from the shake flask. Weight loss was significantly reduced in fermentations (shake flask no 3-6) where carbonic anhydrase was added. To our surprise, adding Zinc (Zn+) to fermentation media improves CO₂ capture significantly. As one can see from the results, the lowest weight loss was seen in flask 5 and 6 so is the ethanol concentration (due to less evaporation loss). The CO₂ capture was further confirmed by observing reagent color. The reagent used consist pH indicator, bromothymol blue in water. The solution turns yellow due to acidic carbonic acid formation after coming into contact with CO₂. In shake flask 1 & 2, the color changed from blue to yellow, indicating CO₂ release from the vent during fermentation. It is interesting to note that the reagent color remained unchanged to blue in the shake flask 5 & 6, where Zn+ was added, indicating no or minimum CO₂ release during fermentation. The reagent color turned from blue to green in shake flask 3 & 4, indicating some release of CO₂ but still less compared to shake flask 1 & 2.

### Example 5B: Effect of Temperature on CO₂ Release

The shake flasks from section 5A were kept in the incubator shaker, and the shaker temperature was increased to 80° C. with an interval of 10° C. every one hour. Temperature was increased to find out optimal temperature for CO₂ release from the fermentation broth. Color of CO₂ check reagent was observed throughout experiment to qualitatively monitor CO₂ release.

TABLE 5B

| Shake | Temperature & color of CO2 check reagent | | | | | |
|---|---|---|---|---|---|---|
| flask | 30° C. | 40° C. | 50° C. | 60° C. | 70° C. | 80° C. |
| 1 | Yellow | Yellow | Yellow | Yellow | Yellow | Yellow |
| 2 | Yellow | Yellow | Yellow | Yellow | Yellow | Yellow |
| 3 | Green | Green | Yellow green | Yellow green | Yellow | Yellow |
| 4 | Green | Green | Yellow green | Yellow green | Yellow | Yellow |
| 5 | Blue | Blue | Yellow green | Yellow green | Yellow | Yellow |
| 6 | Blue | Blue | Yellow green | Yellow green | Yellow | Yellow |

As seen from the results, the optimal temperature of CO₂ release from the captured CO₂ (shake flask 3-6) is ˜80° C. where the reagent color becomes yellow.

The results from 5A and 5B validate our postulates. Carbonic anhydrase captures CO₂ in the fermentation broth while running fermentation. The captured CO₂ is released when the temperature increases to ˜80° C., like the temperature applied in distillation stage.

### Example 6: Probiotic Treated Feedstock, pH Adjustment, Ammonium Nitrate and Carbonic Anhydrase

Finally, lab scale simulation was carried out to see effect of integration of all the above mentioned technologies on ethanol fermentation.

For shake flask 1 and 2, white bread was milled and mixed with water to achieve 30% slurry dry matter. pH was measured (but not adjusted). Liquoflow GO 2× (Dose; 0.2 kg/ton bread) was added in both the shake flask and liquefaction was carried out at 85° C. for 2 hours. The resultant liquefied mashes were cooled down to 32° C. Appropriate quantity of Urea was added in all the shake flasks to achieve 250 ppm inorganic nitrogen concentration. Around 1 gm of dry yeast (Innova Fit) was added in all the shake flasks. Finally, amino glucosidase, Saczyme yield (Dose; 0.5 kg/ton bread) was added in all the flasks.

For shake flask 3 to 6, following procedure was used: Lactobacillus plantarum 1A7 & Lactobacillus fermentum was propagated for 24 h at 30° C. in MRS (Oxoid Laboratories, Hampshire, United Kingdom) with the addition of fresh yeast extract (5% [vol/vol]) and 28 mM maltose to a final pH of 5.6 (mMRS). W. anomalus LCF1694 propagated for 48 h at 30° C. in YEPG (10 g/liter yeast extract, 10 g/liter peptone, 20 g/liter glucose). Appropriate amount of culture from both the species was added to bread flour to achieve around 10\hat{ }3 cells per gm of bread flour. The resultant mixture was mixed with water to achieve 30% slurry dry matter.

For shake flask 7-8, following procedure was used: All bacillus cultures were maintained on 3% trypticase soy broth (TSB) and 0.5% yeast extract agar plates were inoculated into test tubes with 3.5 ml. of 3% TSB and 0.5% yeast extract. Following 24 h of incubation at 37° C. the cultures were transferred to 1 or 2 L volumes of 0.3% TSB in a 1 tube to 1 L ratio. After six days of incubation at 37° C., spores are harvested. Appropriate amount of bacillus spores was added to bread flour to achieve around 10\hat{ }3 spores per gm of bread flour. The resultant mixture was mixed with water to achieve 30% slurry dry matter.

In shake flask no 3 and 4, pH was measured (but not adjusted). In shake flask 5-8 pH was adjusted to 6.20 using 20% ammonia solution. Raw starch hydrolysis enzyme, Saczyme cold mash was added (Dose; 1 kg/ton bread) in the shake flask 3 to 6 and liquefaction was carried out at 45° C. for 30 min. The resultant liquefied mash's were cooled down to 32° C.

Appropriate quantity of urea was added in shake flask no 1 to 4 to achieve 250 ppm inorganic nitrogen concentration. Appropriate quantity of ammonium nitrate was added in shake flask no 5-8 to achieve 250 ppm inorganic nitrogen concentration.

Carbonic anhydrase (Dose: 0.3 kg/ton bread) was added in flask no 5-8 6.

Around 1 gm of dry yeast (Innova Fit) was added in all the shake flasks. Finally, amino glucosidase, Saczyme yield (Dose; 0.5 kg/ton bread) was added in all the flasks. All the flasks were kept in incubator shaker (32° C. at 80 RPM). After 72 hours, sample was taken from the flasks and key parameters like weight loss (escaped CO₂),pH, glucose, ethanol & glycerol were measured.

TABLE 6
Experimental plan

| | | | | Carbonic |
|---|---|---|---|---|
| Shake | Probiotic | pH | Inorganic | anhydrase |
| flask | treatment | adjustment | nitrogen | addition |
| 1 & 2 | no | no | Urea | no |
| 3 & 4 | yes | no | urea | no |
| 5 & 6 | yes | yes | Ammonium nitrate | yes |
| 7 & 8 | yes | yes | Ammonium nitrate | yes |

TABLE 7
Results

| | | | | Final | Ethanol at | Glucose at | Glycerol | Lactic acid at | Acetic acid at |
|---|---|---|---|---|---|---|---|---|---|
| Shake | pH at the | pH at the | Weight | weight of | the end of | the end of | to | the end of | the end of |
| flask | start of the | end of the | loss | the flask | fermentation | fermentation | ethanol | fermentation | fermentation |
| no | fermentation | fermentation | (g) | (g) | (%) | (%) | ratio | (%) | (%) |
| 1 | 5.20 | 4.20 | 11.0 | 64 | 9.50 | 0.10 | 0.10 | 0.08 | 0.05 |
| 2 | 5.20 | 4.15 | 10.50 | 64.50 | 9.48 | 0.09 | 0.12 | 0.05 | 0.06 |
| 3 | 5.20 | 3.50 | 2.50 | 72.50 | 0.80 | 12.0 | 0.35 | 1.1 | 1.5 |
| 4 | 5.20 | 3.20 | 2.10 | 72.90 | 0.78 | 10.5 | 0.38 | 1.0 | 1.3 |
| 5 | 6.20 | 5.85 | 3.15 | 71.85 | 8.60 | 0.05 | 0.09 | 0.50 | 0.65 |
| 6 | 6.20 | 5.90 | 3.00 | 72.0 | 8.58 | 0.03 | 0.08 | 0.42 | 0.58 |
| 7 | 6.20 | 6.00 | 2.95 | 72.05 | 9.05 | 0.05 | 0.07 | 0.07 | 0.1 |
| 8 | 6.20 | 6.05 | 3.05 | 71.95 | 8.95 | 0.02 | 0.07 | 0.06 | 0.09 |

From the above mentioned results, it is seen that

In shake flask 3 and 4, uncontrolled growth of lactobacillus and W. anomalus resulted in ≥100 lactic and acetic acid production which in turn resulted in stressed yeast. Glycerol: ethanol ratio (gig) is a good indicator of yeast stress. Around 0.07-0.09 is considered as healthy ethanol fermentation (Brumm & Hebeda, 1988).

As seen from the table compared to shake flask 1&2, glycerol: ethanol ratio is almost tripled in shake flask 3&4 indicating yeast stress (due to strong inhibitory effect of volatile acids on the yeast). This is also reflected in high unutilized glucose concentration as well as low weight loss.

Surprisingly, despite lactobacillus and W. anomalus growth (confirmed by microscopy), quantity of ethanol produced (g) was improved in shake flask 5 and 6.

Another interesting observation is compared to control (shake flask 1&2) “reduced weight loss” in shake flask no 5-8. Reduced weight loss is an effect of CO₂ capture & converting to carbonic acid by carbonic anhydrase. Moreover, please note that, at industrial scale, significant moisture is lost together with CO₂ as a result of evaporation. It seems that carbonic anhydrase successfully managed to convert CO₂ to carbonic acid and thereby arrested CO₂ and water loss in the atmosphere.

In another observation based on lactic and acetic acid concentration from shake flask 5 &6, it seems that in the beginning both lactobacillus and W. anomalus outgrown yeast, but eventually yeast prevailed. The plausible explanation might be that yeast was not stressed as pH was maintained ≥5.50 due to nitrate respiration and bicarbonate formation (effect of carbonic anhydrase).

In another observation based on bacillus concentration from shake flasks 7&8, it seems that bacillus species did not grow at all in fermentation stage and yeast prevailed. The plausible explanation might be that bacillus is an aerobic organism while ethanol fermentation is strictly anaerobic.

In another observation based on glycerol to ethanol ratio in shake flask 7&8, it seems that yeast cells are not stressed in presence of bacillus which is also reflected in slightly higher ethanol concentration compared to shake flask 5&6. The plausible explanation might be that lactobacillus species can grow in anaerobic environment utilizing some sugars while bacillus can't.

### Example 7: Continuous Fermentation with Three Stages

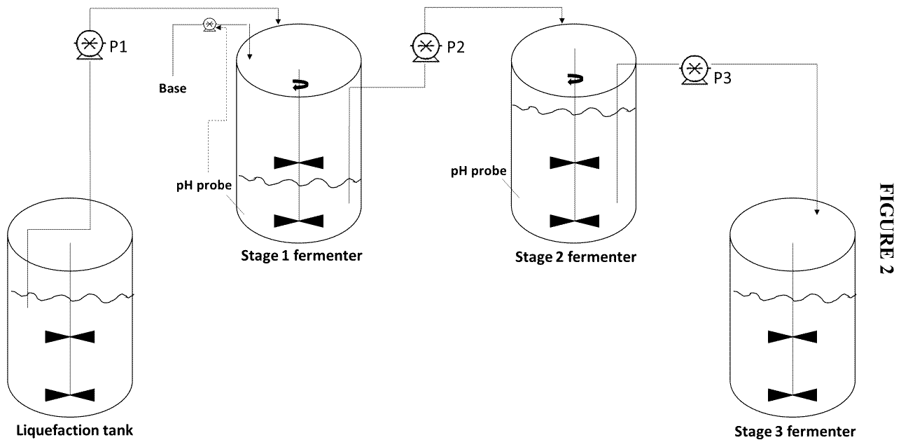

FIG. 2 illustrates an experimental set up of three stage fermentation system. Substrate feed bottle comprised a mixture of bread crump, Saczyme C mash (raw starch hydrolyzing enzyme), Saczyme yield (a mixture of cellulase and glucoamylase), Viscoferm (Xylanase) and urea. The liquefaction was carried out by increasing slurry temperature around 45° C. After two hours, around 1.2 liter of resulting liquefact was transferred to fermenter 1 (stage 1). After adjusting pH to 6.20 using ammonia water, Innova Fit (yeast) was inoculated to kickstart fermentation.

A minimum ethanol concentration of approx. 6% was maintained in order to have bactericidal effect. In order to maintain ethanol in that concentration range, pump P1 & P2 were started with a speed of 150 ml/h when ethanol concentration in fermenter 1 reached ˜6% ethanol (w/w; ˜20 hours). This corresponds to dilution rate (D) of 0.15 h⁻¹. Using cold water, the temperature in stage 1 & stage 2 was maintained at 35° C. & 30° C. respectively. Pump P3 was started with a speed of 150 ml/h when ethanol concentration in fermenter 2 reached ˜10% ethanol (w/w; ˜36 hours). This corresponds to dilution rate (D) of 0.05 h⁻¹. The fermented mash was continuously collected in final fermentation tank (also referred to as the beer well tank) where it was further fermented for another 6-7 hours until reaching in final ethanol concentration of ˜12-15% (w/w). Fermenter levels and pump speed were continuously monitored to avoid any changes in dilution rates. Samples were taken with regular intervals to measure ethanol, sugars, glycerol & biomass, The continuous process ran for ten days to check robustness and to ensure steady states at stage 1 and stage 2.

The same procedure (as mentioned above) was repeated using feedstock pretreated with Probiotics (as described in example 6). Please note that, for this experiment feedstock was treated with Lactobacillus plantarum 1A, Lactobacillus fermentum, W. anomalus and bacilli species.

Finally, a challenge test was performed where Aspergillus niger spores and Lactobacillus fermentum were spiked at stage 1 fermenter to imitate contamination. The main reason behind adding both Aspergillus niger spores and Lactobacillus fermentum is to check the efficacy of continuous fermentation. Aspergillus niger is chosen to represent mold contaminant. Lactobacillus fermentum is chosen to represent a lactic acid bacterium contaminant; it is well known that stuck fermentation can be related to acetic acid production, particularly by obligately heterofermentative species such as L. fermentum and L. mucosae. Samples were taken at regular intervals to perform microscopic examination.

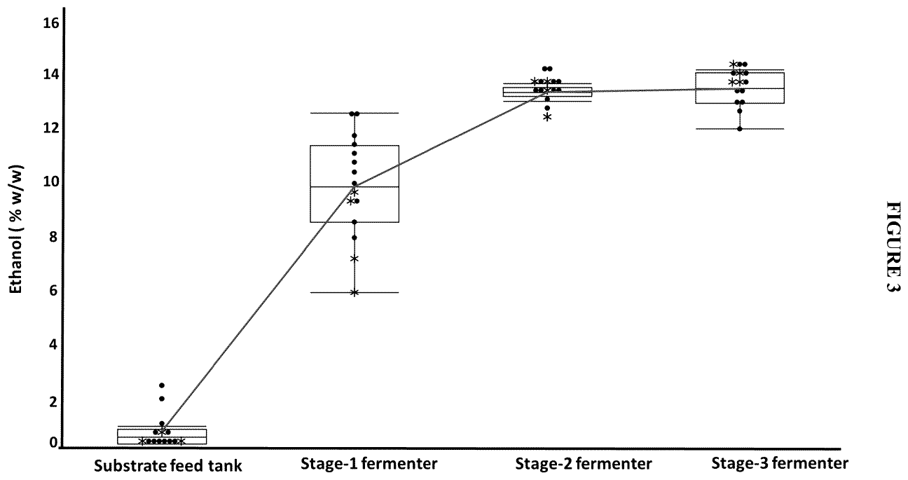

The results from the continuous fermentations can be seen in FIG. 3.

Conclusion: As seen from the FIG. 3, despite contamination (spiking contaminants), ethanol concentration (% w/w) was maintained quite well in the range of 5-8% in stage one and between 10.5-12% in stage 2. Moreover, probiotics (lactobacilli and bacilli species) did not show any adverse effect of ethanol fermentation process.

As seen from the FIG. 3, in stage 1, ethanol concentration was slightly reduced but maintained ≥5%, interestingly the performance was regained in stage 2 indicating robustness of yeast cells to contaminants. In some batches during the challenge test, we observed fungal spores in the sample drawn from stage 1 on day 1 (due to spiking). However, on the second day we did not observe any. A similar observation was made on Lactobacillus fermentum (based on microscopic examination). The possible reason might be unfavorable growth conditions (high concentration of native yeast population means competition for substrate, which is limited, high ethanol concentration and anaerobic condition) resulting in lower (≤0.15 h⁻¹) or no growth (for lactobacillus) and no spore germination (for Aspergillus). These no or very low growth rate resulted in “washed out” as the dilution rate in stage 1 is set at 0.15 h⁻¹. It is well known that in continuous processes, the population which managed to grow at the speed of dilution rate, can able to stay, lower growth rates result in washing out.

The advantage of a continuous system with stage specific growth rate/dilution rate is, it imitates nature's selection mechanism “survival of fittest”. The continuous system ensures contamination prevention due to two selection pressures, high growth rate and high ethanol concentration. Moreover, the stage specific continuous system also ensures healthy population of yeast is always maintained in stage 1 (as soon as growth rate of yeast cell decreases below set dilution rate (0.15 h⁻¹), it gets washed out. Maintaining yeast health plays a critical role towards overcoming possible contamination.

At stage 2, the dilution rate is designed to promote ethanol fermentation. Spores of Aspergillus niger and cells of Lactobacillus fermentum were observed on the same day after spiking in stage 1, confirming “washing out” phenomenon. Moreover, it was also observed that these contaminants got washed out on the next day to beer well tank where it remained inactive.

Stage 3 is beer well tank, its main purpose is to ensure full conversion of starch before sending it to distillation column. The ethanol concentration was measured and found to be in the range of 10-13% w/w.

### Example 8: Sustain^{max} Technology Energy and Water Consumption Overview

Model-based simulation was performed to demonstrate the potential water savings from Cold mash/raw starch hydrolysis step. Thermodynamic based water balance in the state of the art bioethanol plant was used as a baseline (Pfromm. 2008). The water balance is based on fundamental principal, thermal energy intake of state-of-the-art bio-ethanol facilities must be balanced by an appropriate heat sink, here assumed to be evaporation of water in a cooling tower. The model is further extended to include water savings at boiler (as a result of condensate returning to boiler thereby reducing fresh water need in the boiler operations).

The model assumed that average thermal energy consumption in bio-ethanol plant is around 9280 British Thermal Units (BTU) per liter of ethanol is via a boiler where natural gas is combusted and 77% of the liberated heat is transferred to process steam. Thus around 7146 BTU per liter of ethanol produced enter the process. This energy must be discharged back in the environment by evaporating water in cooling tower, which is estimated to be 3.15 l water per 1. ethanol produced (refer to formula no. 1 below). However as there is around 0.32 l.water per l. ethanol contribution from moisture coming from grains, the realized water consumption was found to be 2.8 l.water per 1. ethanol produced. Interestingly G2B Sustain^{Max} process has potential to reduce water consumption to 0.6 l.water/l. ethanol, almost by factor of 5× compared to the traditional process.

This is possibly due to

Energy requirement reduced to 4732 BTU/l ethanol resulting in significant reduction in water required to balance the heat.

Water contribution from grain material is higher than grain used in traditional Bio-ethanol process (as drying is avoided).

Condensate return to boiler resulting in reducing fresh water consumption further.

In the calculations, it was assumed that Ethanol yield and process water remained unchanged in both the process conditions.

Equation to estimate water consumption in ethanol plant:

E_{total,thermal}*ε_{nat.gas}*V_{EtOH}=V_{H20}*pH20(Δh_{latent,H20}+ΔT_{cp} Formula no. 1:

where,
E_{total,thermal} is specific thermal energy from natural gas combustion in Joules per liter ethanol=9692376 J/l. ethanol
ε_{nat.gas} is the thermal efficiency of the process for steam production from combustion of natural gas=0.77
V_{EtOH} is volume of ethanol produced in liters
V_{H20} is volume of cooling water in liters
pH20 is the density of water=1 kg/l
ΔT is 25 KCp is average specific heat capacity of water=4178.2 J/Kg·K
Δh_{latent,H20} is the average enthalpy of vaporization of water=2264000 J/Kg

TABLE 8
Detailed breakdown of variables

| | State of art | | |
|---|---|---|---|
| | ethanol plant | Sustainmax | |
| % Moisture in grain | ~16% | ~20% | |
| material | | | |
| Water contributions | 0.32 | 0.60 | l.water/l.EtOH |
| from grain material | | | |
| Condensate return to | No | Yes | |
| the boiler | | | |
| Water savings in | 0 | 0.60 | l.water/l.EtOH |
| boiler operations | | | |
| (optional) | | | |
| Process water | 0 | 0 | l.water/l.EtOH |
| requirement | | | |
| Total energy | 9194 | 4732 | BTU/l.EtOH |
| requirement | | | |
| Fresh water | 3.15 | 1.57* | l.water/l.EtOH |
| requirement (cooling | | | |
| tower operations) | | | |
| Total water | 2.80 | 0.60** | l.water/l.EtOH |
| consumption | | | |
| Ethanol yield | 0.50 | 0.50 | l.EtOH/kg.DM |

*there is a good chance to reduce water consumption further to ≤0.5 liter as most of the heat injected in the system will be recuperated
**Possible to reduce water consumption to zero

### REFERENCES

Brumm P. J. and Hebeda R. E. Glycerol production in industrial alcohol fermentations. Biotechnology Letters. 1988. Vol 10, No 9, 677-682.
Lewis S. M et. al., Methods and systems for producing ethanol using raw starch. 2011. US007919291B2
Pfromm. P. The Minimum Water Consumption of Ethanol Production via Biomass Fermentation. The Open Chemical Engineering Journal, 2008, 2, 1-5
Rich, J. O., Leathers, T. D., Bischoff, K. M., Anderson, A. M., & Nunnally, M. S. (2015). Biofilm formation and ethanol inhibition by bacterial contaminants of biofuel fermentation. Bioresource Technology, 196, 347-354.
Wu, May, and Xu, Hui. Consumptive Water Use in the Production of Ethanol and Petroleum Gasoline—2018 Update. United States: N. p., 2018.

1. A method for making ethanol and a protein feed or food product, from feedstock comprising starch and protein, said method comprising the steps of:
(a) providing the feedstock comprising starch and protein,
(b) pretreating the feedstock by applying a culture of one or more probiotic species of Lactobacillus and/or spore forming Bacillus to the feedstock,
(c) adding an aqueous liquid to the pretreated feedstock, followed by mixing to obtain a feedstock slurry,
(d) optionally adjusting pH of the feedstock slurry to pH 5-7,
(e) adding an amylase enzyme to the feedstock slurry and incubating the slurry to obtain a feedstock hydrolysate,
(f) adding yeast to the feedstock hydrolysate to obtain a fermentation broth and fermenting the fermentation broth by continuous fermentation comprising two stages, wherein stage 1 has a dilution rate between 0.10-0.55 h⁻¹ and stage 2 has a dilution rate between 0.04-0.10 h⁻¹, resulting in a steady state ethanol concentration between 5-11% (w/w) in stage 1 and between 10-13% (w/w) in stage 2, and
(g) separately recovering
i. ethanol,
ii. CO₂, and
ii. protein,
wherein CO₂ is captured in the fermentation broth during the continuous fermentation by addition of carbonic anhydrase and Zn⁺ to the fermentation broth; and wherein ethanol and CO₂ are separated and recovered in a distillation and condensation step.

2. The method according to claim 1, wherein the feedstock is grain and/or one or more grain derived products.

3. The method according to claim 2, wherein the grain is selected from wheat, rice, oats, barley, rye, barley, millet, corn, triticale, and sorghum grain.

4. The method according to claim 1, wherein the water content of the feedstock is at least 10% w/w, at least 15% w/w, at least 20% w/w, or at least 30% w/w.

5. The method according to claim 1, wherein the probiotic culture in step (b) is applied onto the surface of the feedstock by spraying the probiotic culture on the surface, and optionally mixing.

6. The method according to claim 1, wherein the probiotic culture in step (b) comprises a species of Lactobacillus and a species of spore forming Bacillus.

7. The method according to claim 1, wherein the species of Lactobacillus is selected from Lactobacillus reuteri, Lactobacillus brevis, Lactobacillus plantarum, Lactobacillus amylovorus, and Lactobacillus hammesii.

8. The method according to claim 1, wherein the species of spore forming Bacillus is selected from Bacillus licheniformis, Bacillus clausii, Bacillus subtilis, Bacillus amyloliquefaciens, Bacillus fusiformis and Bacillus megaterium.

9. The method according to claim 1, wherein the continuous fermentation in step (f) comprises an additional third stage having a dilution rate between 0.04-0.10 h⁻¹, resulting in an ethanol concentration in stage 3 between 12-15% (w/w).

10. The method according to claim 1, wherein step (f) comprises maintaining a pH of between 5-6 in stage 1 and stage 2.

11. The method according to claim 1, wherein step (g) comprises addition of 0.3×10⁻¹² to 3.0×10⁻¹² mol Zn⁺ per unit of carbonic anhydrase.

12. The method according to claim 1, wherein the Zn⁺ is added in the form of ZnSO₄.

13. The method according to claim 1, wherein the recovery and separation of the ethanol and the CO₂ comprises vaporizing the ethanol and CO₂, separating the vapors of the ethanol and the CO₂ using a condenser, where the ethanol is condensed in liquid form and the CO₂ is liberated in gaseous form.

14. The method according to claim 13, wherein the temperature for vaporizing the ethanol and CO₂ is 70-90° C., and the temperature in the condenser is 5-30° C.

15. The method according to claim 1, wherein step (g) further comprises recovering an aqueous liquid resulting from one or more preceding steps of the method; and wherein the aqueous liquid added to the feedstock in step (c) comprises the comprises the aqueous liquid recovered in step (g).

16. The method according to claim 15, wherein the aqueous liquid recovered in step (g) is recovered from the distillation step.

17. The method according to claim 15, wherein more than 50% of the aqueous liquid added to the feedstock in step (c) is the aqueous liquid recovered in step (g).

18. The method according to claim 1, wherein the pH in step (d) is adjusted to pH 5.5-7.

19. The method according to claim 1, wherein the pH in step (d) is adjusted using ammonia water, such as ammonia water derived from a biogas plant waste stream.

20. The method according to claim 1, wherein step (e) is performed at a temperatures within the range of 45-55° C.
